(19) United States
(12) Patent Application Publication
(10) Pub. No.: US 2024/0382501 A1
(43) Pub. Date: Nov. 21, 2024

(54) COMBINATION THERAPY FOR TREATMENT OF NEURODEGENERATIVE DISORDERS

(71) Applicant: ALETA NEUROSCIENCE, LLC, MARLTON, NJ (US)

(72) Inventor: Alexander SHTILBANS, Englewood Cliffs, NJ (US)

(21) Appl. No.: 18/788,417

(22) Filed: Jul. 30, 2024

Related U.S. Application Data
(63) Continuation-in-part of application No. PCT/IB2022/053599, filed on Apr. 18, 2022.
(63) Continuation-in-part of application No. 18/490,503, filed on Oct. 19, 2023.
(60) Provisional application No. 63/177,362, filed on Apr. 20, 2021.
(60) Provisional application No. 63/203,819, filed on Jul. 30, 2021.
(60) Provisional application No. 63/261,151, filed on Sep. 14, 2021.
(60) Provisional application No. 63/263,262, filed on Oct. 29, 2021.

(51) Int. Cl. A61K 31/575; A61K 31/122; A61K 31/205; A61P 25/16; A61P 25/28; A61P 37/06

(52) U.S. Cl. CPC A61K 31/575; A61K 31/122; A61K 31/205; A61P 25/16; A61P 25/28; A61P 37/06

(57) ABSTRACT

A method of treating or preventing a neurodegenerative disorder or a peripheral autoimmune disorder is disclosed. The method comprises administering to a patient in need thereof a combination of (a) tauroursodeoxycholic acid (TUDCA) or ursodeoxycholic acid (UDCA), (b) coenzyme Q10 (CoQ10) as ubiquinone or ubiquinol, and (c) creatine. The combination is shown to have synergistic effects in reducing the inflammatory cytokines IL-6 and TNF-α. The combination also significantly increases NFH filament and tubulin filament areas suggesting improved neuronal growth. Unit dosage forms are disclosed. Dosages are disclosed.

### FIELD OF THE INVENTION

Compositions are disclosed to treat and prevent neurological disorders including Alzheimer's disease, Parkinson's disease, Diffuse Lewy Body Disease, Multiple system atrophy, Amyotrophic Lateral Sclerosis, Frontotemporal Dementia, Corticobasal Degeneration, Progressive Supranuclear Palsy and Huntington's disease, and to treat and prevent autoimmune diseases including ankylosing spondylitis, Crohn disease, hidradenitis suppurativa, inclusion body myositis, juvenile idiopathic arthritis, plaque psoriasis, polyarticular juvenile idiopathic arthritis, psoriatic arthritis, rheumatoid arthritis, ulcerative colitis, and uveitis.

### BACKGROUND

Neurodegenerative disorders (ND) are devastating diseases characterized by progressive and irreversible neuronal dysfunction and death [1]. The pathophysiological mechanisms of these diseases are diverse and involve distinct subgroups of neurons in specific areas of the brain [2]. These diseases include: Alzheimer's disease (AD), Parkinson's disease (PD), Diffuse Lewy Body Disease (DLB), Multiple system atrophy (MSA), Amyotrophic Lateral Sclerosis (ALS), Frontotemporal Dementia (FTD), Corticobasal Degeneration (CBD), Progressive Supranuclear Palsy (PSP) and Huntington's disease (HD). These diseases affect many people globally causing severe distress for patients and caregivers, and also result in a large socioeconomic burden [3]. Interestingly, NDs are more prevalent in patients with autoimmune disorders such as rheumatoid arthritis (RA) and inflammatory bowel diseases (IBD). A recent meta analysis evaluating 27 studies showed that individuals with IBD had an increased risk of developing four neurodegenerative disorders: Alzheimer's disease (hazard ratio [HR]=1.35, 95% confidence interval [CI]: 1.03-1.77, P=0.031), dementia (HR=1.24, 95% CI: 1.13-1.36, P<0.001), and Parkinson's disease (HR=1.23, 95% CI: 1.10-1.38, P<0.001) [4]. Two articles included in the meta analysis reported an increased incidence of amyotrophic lateral sclerosis or multiple system atrophy in IBD patients. RA, another autoimmune disease was found to increase risk of dementia by 2.5 folds over 21 years of follow up period [5]. Interestingly, RA drugs inhibiting pathogenetic pro-inflammatory cytokines like tumor necrosis factor (TNF) and interleukin-6 (IL-6) alleviate symptoms such as depression and anxiety [6], [7] and were linked to a reduced risk of future neurodegeneration [8] in RA patients. This suggests a link between peripheral inflammation and diseases of Central Nervous System (CNS). In fact, a comparison of genome-wide association studies (GWAS) on neurodegenerative and chronic immune-mediated diseases revealed shared single-nucleotide polymorphisms (SNPs) related to immune function were also identified for RA and PD, amyotrophic lateral sclerosis (ALS) and progressive supranuclear palsy (PSP) [9].

There is a critical need to develop new and more efficient therapies to combat these prevalent NDs and also to delay onset of these diseases in population at risk. Existing medications can provide improvement for some symptoms and require increased doses as the disease progresses, causing side effects. Despite global research efforts, there are still no neuroprotective or disease-modifying drugs for several ND and nothing is available to prevent them. However, we know that processes leading to neurodegeneration include: a) misfolding of proteins that fail to be cleared from the brain, which in turn stimulates neuroimmune responses; b) calcium excitotoxicity, affecting mitochondria and resulting in energy depletion, leading to neurodegeneration; c) iron accumulation that leads to activation of microglia and further neuroinflammation, causing oxidative stress, formation of reactive oxygen species (ROS), d) mitochondrial dysfunction, and e) neuroinflammation all of which ultimately contributes to neurodegeneration as shown in FIG. 1 [10], [11], [12], [13], [14], [15], All of these processes take place in each patient at different times and different rates. Therefore, it is difficult to test a single medication aimed to improve just one of these processes in a clinical trial using a heterogeneous ND population, as we do not know that all participants have the same degree of dysfunction in a particular targeted process at a given time. One solution is to use personalized medicine, in which we identify patients with the predominantly dysfunctional process and use a medication to repair that. Alternatively, we propose that a “cocktail” of different medications or supplements should be used containing individual agents aimed to address each of the above-mentioned pathways that leads to neurodegeneration in the general patient population of a particular ND. This proposed approach to use a combination of different medications appears to be more realistic, as it could be applied to all patients suffering from that disorder.

The vast majority of basic science and clinical research in the field of ND has been devoted to study individual drugs that can improve single molecular mechanisms leading to neurodegeneration [16]. However, the simultaneous processes leading to neurodegeneration may require a combination of multiple agents to affect the entire disease as it is hard to find just one medication which is able to address all targets in the neurodegenerative cascade, which is ultimately needed to achieve disease-modifying effects in patients. Yet, we know from examples in medicine, such as in HIV and some cancers, that certain conditions can only be controlled when a “cocktail” of different medications is used. However, there has been no rigorous comprehensive evaluation of multiple drug or supplement combinations able to provide an additive disease-modifying effect for NDs.

Additionally, tumor necrosis factor α (TNF-α) may mediate the NDs discussed herein. TNF-α is a central regulator of inflammation in the human body and is also implicated in the pathogenesis of various peripheral autoimmune and inflammatory diseases, including rheumatoid arthritis (RA), and inflammatory bowel diseases (IBD) such as Crohn's disease (CD) and ulcerative colitis (UC). In fact, TNF-α inhibitors, including etanercept (E), infliximab (I). adalimumab (A), certolizumab pegol (C), and golimumab (G), are biologic agents which are FDA-approved to treat such autoimmune disorders as: ankylosing spondylitis (E, I, A, C, and G), Crohn disease (I, A and C), hidradenitis suppurativa (A), juvenile idiopathic arthritis (A), plaque psoriasis (E, I and A), polyarticular juvenile idiopathic arthritis (E), psoriatic arthritis (E, I, A, C, and G), rheumatoid arthritis (E, I, A, C, and G), ulcerative colitis (I, A and G), and uveitis (A). TNF-α is also highly elevated in inclusion body myositis, a neurodegenerative myopathy characterized by accumulation of misfolded proteins, mitochondrial dysfunction and inflammation [18].

### SUMMARY OF THE INVENTION

In an embodiment, this invention provides a method of treating a neurodegenerative disorder selected from at least one of Alzheimer's disease (AD), Parkinson's disease (PD), Diffuse Lewy Body Disease (DLB), Multiple system atrophy (MSA), Amyotrophic Lateral Sclerosis (ALS), Frontotemporal Dementia (FTD), Corticobasal Degeneration (CBD), Progressive Supranuclear Palsy (PSP) and Huntington's disease (HD), or an autoimmune disorder selected from at least one of ankylosing spondylitis, Crohn disease, hidradenitis suppurativa, inclusion body myositis, juvenile idiopathic arthritis, plaque psoriasis, polyarticular juvenile idiopathic arthritis, psoriatic arthritis, rheumatoid arthritis, ulcerative colitis, and uveitis, the method comprising administering to a patient in need thereof a combination of (a) tauroursodeoxycholic acid (TUDCA) or ursodeoxycholic acid (UDCA), (b) coenzyme Q10 (CoQ10) as ubiquinone or ubiquinol, and (c) creatine.

In embodiments, the dosage of TUDCA or UDCA is between 500 mg to 1200 mg per day, the dosage of CoQ10 is between 300 mg and 1200 mg per day, and the dosage of creatine is between 2500 mg and 7500 mg per day. The dosage of each of TUDCA, CoQ10, and creatine may be administered in one extended-release dosage per day, or administered in one to four divided dosages per day. The dosages may be provided as one to six unit dosage forms per administration. Each unit dosage form is selected from a tablet, capsule, or powder for dissolution in a beverage.

### DESCRIPTION OF THE DRAWINGS

FIG. 1 shows multiple processes leading to neurodegeneration. Numbers in parentheses refer to a drug name/class that affects the numbered process: 1. TUDCA; 2. CoQ10; 3. Creatine

FIG. 2 shows the effect of experimental conditions on an iPD-derived iPSC line, a Parkinson's dopaminergic cell line. FIG. 2A shows data for neurofilament heavy chain. FIG. 2B shows tubulin filament data. Statistical analysis: *P<0.05; **P<0.001. FIG. 2A shows cells treated with the combination of TUDCA/Creatine/CoQ10 for 14 days showed 24% increase in NFH filament area compared to the untreated cells (p<0.001). FIG. 2B shows tubulin filament area was also increased by 16% (p<0.001) compared to the untreated cells.

FIG. 3 shows the effect of experimental conditions on MAP2 in a healthy control iPSC line. *P<0.05

FIG. 4 shows the effect of experimental conditions on pro-inflammatory cytokine. FIG. 4A shows interleukin-6 (IL-6) data. FIG. 4B shows tumor necrosis factor-α (TNF-α). *P<0.05; **P<0.001. The triple combination of TUDCA/Creatine/CoQ10 significantly reduced IL-6 activity compared to the individual components, and completely abolished TNF-α activity induced by lipopolysaccharide (LPS) while none of the individual components did.

### DETAILED DESCRIPTION

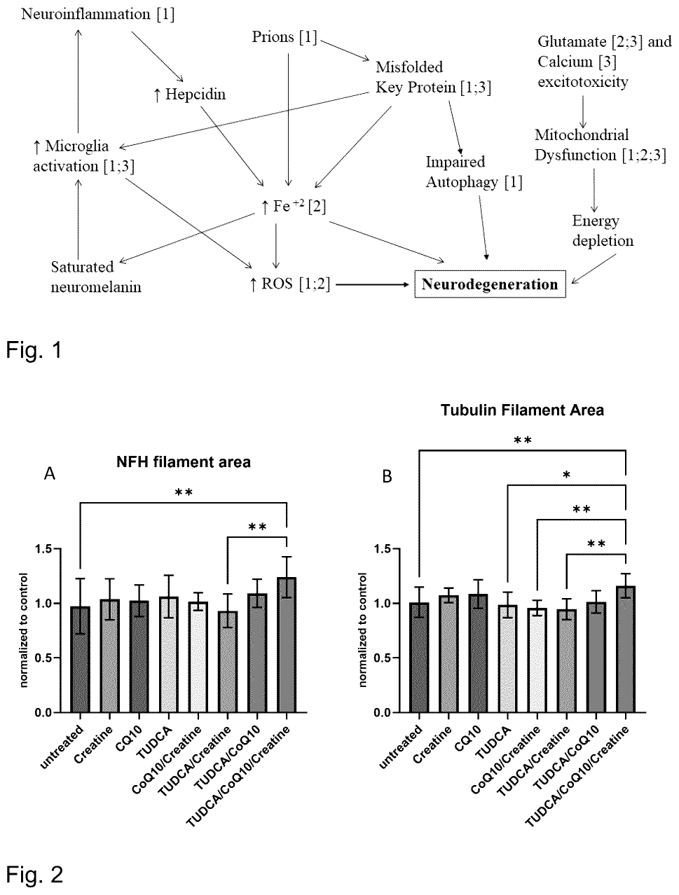

This invention provides pharmaceutical compositions of combinations of three existing nutritional supplements: (a) tauroursodeoxycholic acid (TUDCA) or ursodeoxycholic acid (UDCA), (b) coenzyme Q10 (CoQ10) as ubiquinone or ubiquinol, and (c) creatine. These supplements have been previously evaluated individually in vitro and in vivo for some of the NDs and showed efficacy in preclinical models of ND, but either failed to demonstrate clinical evidence of disease modification/neuroprotection in clinical trials or have not been studied in patients with ND. They were chosen for their different mechanisms of action targeting various processes that lead to neurodegeneration as in FIG. 1. All of these supplements cross blood brain barrier and engage a target as per the published literature: CoQ10 [19], creatine and TUDCA [21]. The combinations as disclosed herein show synergistic effects and may be effective in treating and preventing the neurodegenerative and autoimmune diseases mentioned herein.

This invention further provides a method of treating or preventing a neurodegenerative disorder selected from at least one of Alzheimer's disease, Parkinson's disease, Diffuse Lewy Body Disease, Multiple system atrophy, Amyotrophic Lateral Sclerosis, Frontotemporal Dementia, Corticobasal Degeneration, Progressive Supranuclear Palsy, Huntington's disease, or an autoimmune disorder selected from ankylosing spondylitis, Crohn disease, hidradenitis suppurativa, juvenile idiopathic arthritis, plaque psoriasis, polyarticular juvenile idiopathic arthritis, psoriatic arthritis, rheumatoid arthritis, ulcerative colitis, uveitis, and inclusion body myositis. In the method, a combination of (a) TUDCA or UDCA, (b) CoQ10 and (c) creatine are administered to a patient suffering from one of these neurodegenerative disorders or peripheral autoimmune disorders, or at risk of developing one of these neurodegenerative disorders or peripheral autoimmune disorders.

A number of specific processes are implicated in neurodegenerative diseases. FIG. 1 is a diagram showing relationships believed to lead to neurodegeneration in disorders discussed herein. Starting from the top right of FIG. 1, glutamate and calcium excitotoxicity can lead to mitochondrial dysfunction which leads to energy depletion and neuronal death [22]. In excitotoxicity, nerve cells suffer damage or death when the levels of otherwise necessary and safe neurotransmitters such as glutamate become pathologically high, resulting in excessive stimulation of receptors. For example, when glutamate receptors such as the NMDA receptor or AMPA receptor encounter excessive levels of the excitatory neurotransmitter glutamate, significant neuronal damage may ensue. Excess glutamate allows high levels of calcium ions (Ca²⁺) to enter the cell. Highly elevated intraneuronal calcium levels are implicated in mitochondrial dysfunction and the production of reactive oxygen species leading to apoptosis.

Additionally, overaccumulation and misfolding of key proteins in the brain coupled with impaired autophagy leads to neurodegeneration. Moreover, misfolded proteins lead to over-accumulation of intracellular iron (II) in the brain which in turn leads to formation of reactive oxygen species (ROS) and neurodegeneration [23], [24]. The over-accumulated iron is removed from the brain by the natural iron chelator neuromelanin [25], [26]. Once neuromelanin is saturated with iron, it causes activation of microglia which leads to formation of more ROS as well as neuroinflammation which in turn results in more brain iron accumulation closing the feedback loop.

The combinations disclosed herein were evaluated for their effect in an in-vitro Parkinson's Disease model. The diversity of the simultaneously occurring processes leading to neurodegeneration in each individual patient with a neurodegenerative disorder cannot be addressed with just one medication. Various combinations of potentially neuroprotective agents targeting different disease mechanisms simultaneously may show improved additive or synergistic efficacy in slowing the disease progression and allow agents to be utilized in humans at lower doses to minimize side effects. An iPSC cell-derived human dopaminergic neurons from a patient with Parkinson's Disease and healthy control as well as microglial cells were used to evaluate effects of these combinations on neurodegeneration and neuroinflammation. As metrics, we used neurofilament heavy chain (NFH), tubulin, microtubule-associated protein 2 (MAP2) and pro-inflammatory cytokines.

The data shows that combinations disclosed here exerted a synergistic neuroprotective effect in preclinical models of a neurodegenerative disease that is significantly superior to each of the compounds individually. Because TUDCA, CoQ10, and creatine are individually well known, they can be administered to patients with or at risk for neurodegenerative disorders with little to no risk. The profound inhibitory effect of the described combination on TNF-α may also be useful for treatment of peripheral autoimmune diseases.

### Bile Acids

Tauroursodeoxycholic acid (TUDCA), an endogenous bile acid, is a strong neuroprotective agent. TUDCA is a taurine conjugate of ursodeoxycholic acid (UDCA). It is permeable to the blood-brain barrier and has a low toxicity profile [28], [29]. TUDCA has been shown to have beneficial effects in AD, ALS, PD and HD. These disorders share the pathologies of accumulation of protein aggregates in the brain, neuroinflammation and mitochondrial dysfunction [30]. Bile acids such as UDCA and TUDCA have been shown to suppress the toxic aggregation of misfolded proteins in various animal models of neurodegenerative diseases. These bile acids safeguard neurons also by reducing the synthesis of reactive oxygen species, mitigating mitochondrial damage, and inhibiting apoptosis through both the intrinsic and extrinsic pathways [28]. Additionally, TUDCA showed anti-inflammatory effect in multiple pre-clinical models of neurodegenerative diseases [31-34]. Moreover, TUDCA and UDCA substantially reduced PrP conversion in cell-free aggregation assays, and in chronically and acutely infected cell cultures. TUDCA and UDCA also reduced neuronal loss in prion-infected cerebellar slice cultures suggesting they may have a therapeutic role in prion diseases [35].

### Alzheimer's Disease

TUDCA inhibits the accumulation of amyloid β (Aβ) deposits in AD. It also prevents microglial activation and a loss of neuronal integrity. Connective tissue growth factor (CTGF) is a cysteine-rich protein that has been shown to promote the activity of γ-secretase and Aβ neuropathology [36]. TUDCA can conflict with the processing of APP and it has an inhibitory effect on the expression of CTGF. TUDCA decreases the production of amyloidogenic APP-CTF-γ and APP-CTF-β, direct precursors of A⊖. TUDCA or similarly acting compounds such as UDCA could be therapeutically useful γ-secretase modulators [36], [37]. In APP/PS1 mice, an experimental model of AD, a 6-month treatment with 0.4% of TUDCA in diet prevented Aβ plaque accumulation in the brain [36], [37]. An improvement in the spatial, recognition and contextual memory was also observed in APP/PSI mice after this treatment.

The fundamental basis of TUDCA's neuroprotective ability is more focused on its anti-apoptotic properties than on ER stress relieving activity. TUDCA exerts anti-apoptotic effects by minimizing nuclear fragmentation; by reducing caspase 2 and 6 activations; and by modulating p53, Bcl-2, and Bax activity [38]. The treatment with 100 μM of TUDCA for 12 h can significantly decrease Aβ peptide-associated apoptosis in cortical neurons [39].

Therefore, both in vitro and in vivo results show the effectiveness of TUDCA in decreasing apoptosis, attenuating Aβ production and deposition, Tau hyperphosphorylation, and loss of synaptic function.

### Parkinson's Disease

Duan et al showed that the application of TUDCA facilitates the survival of DA neurons in vitro and in vivo conditions [40]. TUDCA-treated group demonstrated increase in the number of tyrosine hydroxylase positive neurons, used as a marker for dopamine, norepinephrine, and epinephrine-containing neurons [41]; and a reduction in the number of apoptotic cells. In MPTP mouse model, pre-treatment with TUDCA (50 mg/kg for 3 days) significantly reduced neurodegeneration of the nigral dopaminergic neurons caused by MPTP, as well as reduced dopaminergic fiber loss and ameliorated motor performance and symptoms typical of PD, such as spontaneous activity, ability to initiate movement and tremors. [42]. In the same study, TUDCA treatment also prevented the production of MPTP-dependent ROS [42]. In chronic mouse model of PD, pretreatment with TUDCA prevented protein oxidation and autophagy, in addition to inhibiting alpha-SYN aggregation [43]. TUDCA-dependent mitoprotective effects have also been observed in primary mouse cortical neurons and neuroblastoma cell line SH-SY5Y [44]. All this makes TUDCA useful in attenuating mitochondrial dysfunction and ROS production as well as inhibiting multiple proteins involved in apoptosis.

### Amyotrophic Lateral Sclerosis

In a small double-blind, placebo-controlled study of TUDCA in riluzole-treated patients, TUDCA was well tolerated. The proportion of responders was higher with TUDCA (87%) than with placebo (P=0.021; 43%). At study end baseline-adjusted ALSFRS-R was significantly higher (P=0.007) in TUDCA than in placebo groups. Comparison of the slopes of regression analysis showed slower progression in the TUDCA than in the placebo group (P<0.01) [45]. TUDCA in combination with sodium phenylbutyrate has also been found to reduce reactive oxygen metabolite-mediated oxidative damage in neurons and improve neuronal viability [46]. This combination is now FDA-approved for treatment of ALS.

### Huntington's Disease

The treatment with TUDCA exhibited a significant reduction in apoptosis in a 3-NP rat model of HD, as well as preserved striatal mitochondria morphology [47]. In cultured striatal cells, TUDCA treatment prevented 3-NP-mediated neuronal death [47]. The treatment with 500 mg/kg of TUDCA also generated neuroprotective effects in the R6/2 transgenic mice model of HD [48]. TUDCA-treated mice exhibited significant improvement in locomotor and sensorimotor deficits.

### Autoimmune Diseases

Numerous research studies have investigated the potential therapeutic effects of TUDCA in managing autoimmune conditions. Van den Bossche et al. (2017) investigated the effects of TUDCA on bile acid homeostasis and inflammation in an experimental model of Crohn's disease (CD) [49]. The study analyzed gene expression of nuclear receptors and bile acid transporters in Caco-2 cell monolayers exposed to tumor necrosis factor (TNF)α, ileal tissue of TNFΔARE/WT mice, and inflamed ileal biopsies from CD patients. The results showed that TUDCA counteracted the downregulation of nuclear receptors and bile acid transporters caused by TNFα exposure. In TNFΔARE/WT mice, TUDCA administration reduced ileitis severity and preserved bile acid homeostasis, indicating the TUDCA's efficacy in experimental colitis and potential as a therapeutic agent for inflammatory bowel disease (IBD). Previously, Yang et al. (2016) evaluated the anti-inflammatory effects of TUDCA in a mouse model of colitis induced by 2,4,6-trinitrobenzenesulfonic acid (TNBS). The study found that TUDCA significantly improved body weight, decreased macroscopic and histopathological scores, and reduced levels of MPO activity, IL-1β, IFN-γ, and TNF-α in colonic tissue [50]. These findings indicate that TUDCA alleviates colitis symptoms and possesses significant anti-inflammatory properties in TNBS-induced UC, suggesting its potential as a therapeutic agent for UC. Additionally, Zhao et al. (2024) demonstrated the therapeutic potential of TUDCA/Emodin liposomes in treating ulcerative colitis (UC) [51]. The study prepared TUDCA/Emodin liposomes and assessed their effects on UC symptoms. After oral administration, TUDCA/Emodin liposomes significantly alleviated UC severity in mice, evidenced by increased colon length, decreased inflammation, and reduced colonic ER stress. They also restored the integrity of the intestinal barrier and promoted gut microbiota restoration.

### CoQ10

Coenzyme Q10 (CoQ10), also known as ubiquinone, is produced in the body and is implicated in many cellular processes. The presence of CoQ10 is not limited to mitochondria but extends to all cellular membranes and blood, highlighting its importance in animal and plant cells. Additionally, CoQ10 inhibits glutamate excitotoxicity and oxidative stress-mediated mitochondrial alteration in a mouse model of glaucoma [52]. Levels CoQ10 are known to decrease with age in both animals and humans, indicating its significance in age-related neurodegenerative diseases [53]. Ubiquinol, the reduced form of ubiquinone, is believed to be the metabolically active form of CoQ10. The administration of ubiquinone or ubiquinol are within the scope of this invention.

### Alzheimer's Disease

As a crucial component of the mitochondrial electron transport chain, CoQ10 has demonstrated promising roles in preclinical neuroprotective interventions for neurodegenerative diseases, particularly Alzheimer's disease. Studies have shown that CoQ10 can reduce amyloid beta (Aβ) plaque burden in transgenic mouse models of AD, both intracellularly and in terms of plaque pathology, by oral administration, suggesting its ability to cross the blood-brain barrier [55]. In another study, CoQ10 has been shown to protect against Aβ-induced impairment in hippocampal long-term potentiation, a key process in learning and memory, further underscoring its neuroprotective effects through antioxidant activity [56]. Moreover, its antioxidant properties have been linked to improvements in cognitive function and reductions in oxidative damage in the hippocampus and cerebral cortex of rat models [57]. Additionally, CoQ10 has shown efficacy in reducing oxidative stress and amyloid pathology, improving behavioral performance, and decreasing brain levels of protein carbonyls, a marker of oxidative stress, in AD mouse models [55]. Furthermore, a water-soluble formulation of CoQ10 has been effective in stabilizing mitochondria, preventing neuronal cell death, and reducing circulating Aβ peptide in AD mouse models, suggesting its potential to inhibit disease progression [58]. Furthermore, the water-dispersible, nanomicellar formulation of CoQ10, Ubisol-Q10, have shown promise by enhancing bioavailability and demonstrating neuroprotective effects against neurotoxin exposure in both in vitro and in vivo neurodegeneration models. This formulation has been recognized for its various neuroprotective mechanisms, including as an antioxidant, senescence preventer, autophagy activator, anti-inflammatory agent, and mitochondrial stabilizer [59]. In human clinical trials, CoQ10 has also shown potential in treating Huntington's disease and Parkinson's disease, supporting its use in neurodegenerative conditions where mitochondrial dysfunction and oxidative damage are implicated [60].

### Parkinson's Disease

In PD, a study with different dosages of CoQ10 showed that higher doses might slow disease progression, indicated by a lesser increase in scores on the Unified Parkinson Disease Rating Scale (UPDRS) [61]. Various formulations of CoQ10 have been tested for their neuroprotective effects against dopaminergic neuron loss in PD models, revealing that CoQ10, especially when reduced (ubiquinol), can significantly increase plasma concentrations and provide neuroprotection [62]. Interestingly, prophylactic application of water-soluble CoQ10 formulations in rat models showed promising results in preventing dopaminergic neurodegeneration and behavioural impairment [63].

Another study showed the potential of prophylactic and therapeutic CoQ10 treatment in a PQ-induced PD mouse model. They reported that CoQ10 effectively reduced protein carbonyl content in the brain and improved behavioural outcomes [64]. A preclinical study determined a neuroprotective role of coenzyme Q10 in iron-induced apoptosis in cultured human dopaminergic (SK-N-SH) neurons, in metallothionein gene-manipulated mice, and in alpha-synuclein knockout (alpha-synko) mice with a primary objective to assess a possible therapeutic potential for CoQ10 in PD [65]. Another study demonstrated that continuous, intrastriatal administration of CoQ10 could prevent dopaminergic neuron degeneration more effectively than oral administration, suggesting a new strategy for neurodegeneration prevention in PD [66]. However, high doses of CoQ10 failed to show any disease-modifying effects in a randomised, double-blind placebo-controlled clinical trial in PD [67].

Interestingly, studies have shown that CoQ10, combined with other supplements like creatine, can provide additive neuroprotective effects in various neurodegenerative disease models. This combination has been effective in reducing oxidative stress, DNA damage, and neurodegeneration in animal models of PD and Huntington's disease [68].

### Huntington's Disease

In the case of Huntington's disease, some studies, particularly those using juvenile mice model, have indicated that CoQ10 can improve symptoms and prolong survival in HD. Research using the CAG140 knock-in mouse model, which carries an expanded CAG repeat mimicking the human HD mutation, showed improvement in early behavioral deficits and some transcriptional disturbances, without affecting huntingtin aggregates in the striatum. Also, they found that a lower CoQ10 dose (0.2%) was found to be more effective than a higher dose (0.6%). They suggested the benefits of CoQ10 in HD may be maximized when treatment is initiated in the early stages of the disease and that the dosage is critical [69]. Further investigation into the HD gene mutation reveals a potential primary or secondary impact on energy metabolism, as evidenced by decreased phosphocreatine to inorganic phosphate ratios and increased lactate concentrations in the cerebral cortex of HD patients. CoQ10 treatment significantly reduced cortical lactate concentrations, indicating at a general energy deficit in HD and suggesting a possible therapeutic role for CoQ10 [70]. Moreover, CoQ10's effectiveness, in conjunction with the NMDA antagonist remacemide, extended survival and delayed the onset of motor deficits, weight loss, and other pathological features in transgenic mouse models of HD. This suggests that addressing bioenergetic defects and excitotoxicity, both implicated in HD pathogenesis, can be an effective therapeutic strategy [72]. Based on these research studies, CoQ10's role in neurodegenerative conditions indicate that it might help ameliorate symptoms, improve patient's condition and enhanced quality of life.

### Autoimmune Diseases

Abdollahzad et al. (2015) reported a double-blind, randomized controlled trial to assess CoQ10→s impact on cytokine production and oxidative stress in rheumatoid arthritis (RA) patients [73]. Forty-four RA patients were assigned to receive either 100 mg/day CoQ10 or a placebo for two months. The outcomes showed that CoQ10 significantly reduced serum malondialdehyde (MDA) levels and suppressed tumor necrosis factor-alpha (TNF-α) overexpression. These findings suggest CoQ10 effectively reduces oxidative stress and inflammatory cytokines, highlighting its potential as an adjunctive therapy in managing RA. Additionally, Nachvak et al. (2019) evaluated the effect of CoQ10 on serum matrix metalloproteinases (MMP) levels and clinical parameters in RA patients in a randomized, double-blind, placebo-controlled trial [74]. Both groups showed significant reductions in serum MMP-1 levels, swollen joint count, and DAS-28 scores. However, serum MMP-3 levels significantly increased in the placebo group. Notably, CoQ10 also reduced the erythrocyte sedimentation rate (ESR), pain score, and tender joint count. These results indicate CoQ10's potential in reducing serum MMP-3 levels and improving clinical outcomes in RA patients. Furthermore, Farsi et al. (2021) evaluated the effects of CoQ10 in Eighty-eight patients with ulcerative colitis (UC) [75]. The CoQ10 group showed significant reductions in Simple Clinical Colitis Activity Index (SCCAI) scores, diastolic and systolic blood pressure, and significant improvements in Inflammatory Bowel Disease Questionnaire-32 (IBDQ-32) scores. These findings suggest that CoQ10 can effectively reduce disease severity and improve quality of life in UC patients. In another study, El Morsy et al. (2015) explored the protective effects of CoQ10 and amlodipine on UC in a rat model [76]. The results showed that CoQ10 and amlodipine, individually and combined, significantly reduced colon tissue MDA, TNF-α, IL-1β, PGE2, MPO, and HSP70 levels, while increasing SOD activity, ATP, and IL-10 levels. Also, the histological examination showed restoration of colon structure. Similarly, Ewees et al. (2016) also reported beneficial outcomes of CoQ10 during an investigation against UC induced by iodoacetamide in rats [77]. CoQ10 significantly increased catalase activity and glutathione content. It also significantly decreased myeloperoxidase activity, malondialdehyde content, and nitrate/nitrite production, suggesting reduced oxidative stress and inflammation. These findings suggest that CoQ10 alone or in combination with standard therapy can be a valuable adjunctive treatment for UC, offering antioxidant, anti-inflammatory, and energy restoration properties.

Al-Oudah et al. (2022) evaluated the effects of CoQ10 as an adjuvant therapy in Twenty-four Iraqi psoriatic patients [78]. The study found significant correlations between Psoriasis Area and Severity Index (PASI) and Dermatology Life Quality Index (DLQI) scores. The CoQ10 group showed a 67.48% improvement in PASI scores and a 56.13% improvement in DLQI scores. These findings suggest that CoQ10, as adjunctive therapy, significantly improves clinical severity and quality of life in psoriasis patients.

### Creatine

Creatine (CR), commonly recognized as an ergogenic aid for sports and exercise, is showed promising neuroprotective properties against different neurodegenerative conditions in preclinical models. Recent studies reported its cognitive enhancement potential in both younger and older adults [79].

### Alzheimer's Disease

Studies on Alzheimer's disease rodent models suggested that CR supplementation could improve mitochondrial function and offer neuroprotective benefits [80, 81]. It offers prevention and symptomatic treatment in AD models, indicating its role beyond physical performance enhancement [82]. Creatine protected cultured primary embryonal hippocampal and cortical cells against glutamate and calcium excitoxicity in an in vitro model of neurodegeneration [83]. In another study using the 3xTg mouse model of AD, creatine supplementation (3% w/w) over 8-9 weeks revealed notable improvements in spatial cognition in female mice. This improvement was accompanied by enhancements in mitochondrial function, increased phosphorylation of CREB, and changes in levels of proteins related to plasticity and AD pathology, including a rise in IκB (NF-κB suppressor), CaMKII, PSD-95, and high-molecular-weight amyloid β (Aβ) species. Interestingly, Aβtrimers were reduced, indicating a possible mechanism through which creatine exerts its effects on AD pathology [80]. Also, creatine supplementation mitigated spatial learning and memory deficits in young female rats. The study reported that creatine decreased latency and errors in a Barnes maze test and improved recognition memory in a novel object recognition test. The positive cognitive effects were linked to upregulated mTORC1 signaling and enhanced synaptic proteins like synapsin and PSD-95 within the dentate gyrus, suggesting a role in enhancing synaptogenesis and signaling pathways related to memory and learning [84]. Moreover, a study in elderly population showed that 5 g creatine four times daily for a week showed significant improvements in cognitive tasks, including random number generation, forward and backward number and spatial recall, and long-term memory tasks. This suggests that creatine may aid cognitive processing in the aging brain [85].

### Parkinson's Disease

In PD, a clinical trial examined the combined effect of creatine and coenzyme Q10 (CoQ10) on mild cognitive impairment in PD (PD-MCI). The patients were randomly treated with creatine monohydrate (5 g b.i.d.) and CoQ10 (100 mg t.i.d.) or a placebo. The study found that after 12 and 18 months, the combination therapy group showed significant improvements in cognitive scores and reduced plasma phospholipid levels compared to the control group, suggesting a neuroprotective function of this therapy in PD-MCI patients [86]. Another study focusing on the therapeutic effects of resistance training with and without creatine supplementation in patients with mild to moderate PD revealed that creatine supplementation enhanced the benefits of resistance training. This was evident in increased chest press and biceps curl strength and improved chair rise performance, indicating improved muscular fitness and functionality in PD patients [87]. However, high doses of creatine failed to show any disease-modifying effects in a long, randomised, double blind placebo-controlled clinical trial in PD [88].

Moreover, in a mouse model of PD, the combined administration of cyclooxygenase 2 (COX-2) inhibitor rofecoxib and creatine showed significant neuroprotective properties. They effectively protected against striatal dopamine depletions and loss of substantia nigra tyrosine hydroxylase immunoreactive neurons, suggesting the potential of creatine as part of a combined therapeutic strategy for PD [89]. Furthermore, creatine and exercise had anti-inflammatory and antioxidative effects in MPTP mice, as evidenced by a decrease in microglia activation [90].

### Huntington's Disease and Amyotrophic Lateral Sclerosis

Furthermore, the role of creatine supplementation in ALS has been explored through various studies. In a research study of 28 ALS patients, 20 g of creatine daily for 7 days, then 3 g for 6 months temporarily increased muscle power and strength. However, all parameters declined progressively over the 6-month follow-up period. This study suggests that creatine supplementation may temporarily enhance muscular power in ALS patients, potentially offering symptomatic relief during high-intensity activities [91]. Another study on transgenic ALS mice with SODI mutations showed that creatine improved motor performance, extended survival, and protected against loss of motor and substantia nigra neurons. The study indicated that creatine stabilizes mitochondrial creatine kinase and inhibits the opening of the mitochondrial transition pore, suggesting a therapeutic potential for ALS treatment [92]. In a study using G93A transgenic mice, a model for familial ALS, the impact of long-term creatine supplementation was assessed. Creatine supplementation showed promising results, improving both lifespan and motor function in these mice. Notably, creatine helped reduce glutamate levels at 75 days of age but did not have the same effect later at 115 days. This suggests that the benefits of creatine might be linked to its support of glutamate transporter function, which is highly energy-dependent and vulnerable to oxidative damage [93].

Moreover, studies have demonstrated that dietary creatine supplementation can significantly improve both clinical and neuropathological outcomes in transgenic Huntington disease mouse models. The supplementation of creatine extended survival significantly in the R6/2 transgenic mouse model of HD, particularly when started at early or middle stages of the disease. This treatment also improved motor performance, reduced body weight loss, and delayed neuropathological manifestations such as brain and neuronal atrophy, as well as huntingtin aggregates [94]. Similarly, another study found that dietary supplementation with 2% creatine markedly improved survival, motor symptoms, and weight loss in N171-82Q transgenic mice. It also attenuated brain atrophy, intranuclear inclusion formation, and reductions in striatal N-acetylaspartate, suggesting an overall neuroprotective effect [95]. Also, a study confirmed that dietary creatine supplementation in HD mice improved survival, delayed brain atrophy and striatal neuron degeneration, and enhanced motor performance. A small study in twenty patients with Huntington's Disease demonstrated that Creatine supplementation reduced brain glutamate [96].

### Autoimmune Diseases

Limited studies indicate the potential of creatine in managing autoimmune diseases. In a randomized controlled trial, Wilkinson et al. (2016) investigated the impact of oral creatine (Cr) supplementation on rheumatoid arthritis (RA) patients [97]. The 40 RA patients were suffering from rheumatoid cachexia, characterized by muscle wasting, reduced strength, and impaired physical function. The outcomes showed that Cr supplementation significantly increased appendicular lean mass (ALM) by 0.52±0.13 kg (P=0.004) and total lean mass (LM) by 0.60±0.37 kg (P=0.158), correlated with a gain in intracellular water (0.64±0.22 liters; P=0.035). However, there were no significant improvements in isometric knee extensor strength, handgrip strength, or physical function. The study concluded that Cr supplementation effectively increases muscle mass without enhancing strength or physical function, indicating its potential as a safe adjunct treatment for muscle loss in RA patients experiencing severe rheumatoid cachexia. In a recent case report, Roy and Lee (2016) evaluated the potential therapeutic benefits of creatine in inflammatory bowel disease (IBD), specifically Crohn's ileitis [98]. The patient with IBD experienced significant clinical improvement with creatine supplementation. Initially diagnosed with mild Crohn's ileitis, the patient was treated with mesalamine but stopped taking creatine, resulting in worsened symptoms and increased ileitis and ulceration. After restarting creatine hydrochloride supplementation, significant symptom improvement was observed, and a follow-up colonoscopy six months later showed substantial improvement in mucosal ulceration and inflammation. These findings suggest that creatine supplementation could play a beneficial role in maintaining intestinal mucosal function and mitigating inflammation in IBD patients.

To test all combinations of the three supplements above for possible additive/synergistic effects, we used in vitro PD and Neuroinflammation models as examples. We studied effects of these combinations on iPSC cells derived from dopaminergic neurons of a patient with idiopathic PD and of a healthy control. We then used human microglial cells to test the same combinations for potential anti-neuroinflammatory effects.

### Combinations and Dosages

In an embodiment, this invention provides for combinations of TUDCA or UDCA, CoQ10, and creatine as a pharmaceutical composition for the treatment or prevention of neurological disorders. In an embodiment, the dosages of each of these supplements is: TUDCA, between 500 mg to 1200 mg per day; CoQ10, between 300 mg and 1200 mg per day (or ubiquinol 200-1000 mg); and creatine, between 2500 mg and 7500 mg per day.

In an embodiment, the combinations of this invention may be administered once per day in an extended-release formulation, or two to four times per day in divided doses.

The combinations may be formulated into unit dosage forms containing all three supplements. Exemplary unit dosage forms include tablets, capsules, and powders for dissolution in a beverage. Exemplary beverages include soft drinks including carbonated beverages, fruit juices, vegetable juices, other flavored drinks, and water.

In an embodiment, the unit dosage forms may be provided as one to six tablets or capsules intended for administration at one time up to four times a day, for example, four tablets given three times per day.

### Formulations

Actual dosage levels of the active ingredients (TUDCA or UDCA), CoQ10, and creatine) in the pharmaceutical compositions of the invention may be varied so as to obtain an amount of the active ingredient that is effective to achieve the desired therapeutic response for a particular patient, composition, and mode of administration, without being toxic to the patient.

In an embodiment, the pharmaceutical or cosmetic solid compositions of the invention can be formulated in any form that includes a single or multiple unit dosage form. The term “single unit” encompasses one entity such as a single tablet, a single capsule, a single granule, a powder and a single pellet that contains all active ingredients (TUDCA or UDCA, CoQ10, and creatine) in the tablet, capsule, or powder. The term “single unit dosage form” defines a dosage form which consists only of one unit which contains the effective amount of all active ingredients. The term “multiple unit dosage form” defines a dosage from which consists of more than one unit which contains all active ingredients. Multiple unit dosage forms may be used, for example, to reduce the size of any given unit dosage form such as a tablet or capsule, since large tablets and capsules may be hard for many patients to swallow. For example, a total dose of active ingredients in this invention may be 5000 mg/day. Divided into three equal dosages, that is 1666.66 mg per dose. A single tablet containing 1666.66 mg may be difficult to swallow for many persons. Thus, further dividing the dose into three tablets of 555.55 mg each, or four tablets of 416.66 mg each may be easier to swallow. These sets of three or four tablets are a multiple unit dosage form given at one time to a patient.

In an embodiment, the composition is a capsule; for example a hard capsule. In an embodiment, the solid composition is a tablet; for example a direct-compressed tablet or a dry-granulation tablet. In an embodiment, the pharmaceutical or cosmetic solid composition is an immediate release composition. In an embodiment, the pharmaceutical or cosmetic solid composition is a extended release composition. The term “extended release” refers to a composition in which the rate of release of the active ingredients from the composition after administration has been changed by the addition of excipients that delay the release of the active ingredients in the gut, allowing for fewer dosages per day, for example once per day instead of two to four divided doses. The term “extended release” as used herein includes a controlled release, a sustained release, or a prolonged release formulation.

The pharmaceutical composition as defined above may further comprise appropriate excipients or carriers including, but not limited to, binders, fillers, disintegrants, glidants, lubricants or their mixtures. Additionally, the compositions of the present invention may contain other ingredients, such as colorants, and other components known in the state of the art for use in pharmaceutical and cosmetic compositions.

The term “binder” refers to any pharmaceutically acceptable compound having binding properties. Materials commonly used as binders include povidone such as polyvinylpyrrolidone, methylcellulose polymers, hydroxyethyl cellulose, hydroxypropyl cellulose, L-hydroxypropyl cellulose (low substituted), hydroxypropylmethyl cellulose (HPMC), sodium carboxymethyl cellulose, carboxymethylene, carboxymethylhydroxyethyl cellulose and other cellulose derivatives, starches or modified starches, gelatine, sugars such as sucrose, glucose and sorbitol, gums such as sum arabic, tragacanth, agar and carragenenan; and mixture thereof. In an embodiment, the composition of the invention is one wherein the pharmaceutically or cosmetically acceptable excipients or carriers comprise one or more binder; preferably comprise polyvinylpyrrolidone. In an embodiment, the composition of the invention is one wherein the pharmaceutically or cosmetically acceptable excipients or carriers comprise one or more binder in an amount from 1% to 10% by weight, preferably from 1% to 6% by weight, more preferably from 1% to 3% by weight of the composition.

The terms “filler” and “diluent” have the same meaning and are used interchangeably. They refer to any pharmaceutically acceptable excipient or carrier (material) that fill out the size of a composition, making it practical to produce and convenient for the consumer to use. Materials commonly used as filler include calcium carbonate, calcium phosphate, dibasic calcium phosphate, tribasic calcium sulfate, calcium carboxymethyl cellulose, cellulose, cellulose products such as microcrystalline cellulose and its salts, dextrinderivatives, dextrin, dextrose, fructose, lactitol, lactose, starches or modified starches, magnesium carbonate, magnesium oxide, maltitol, maltodextrins, maltose, mannitol, sorbitol, starch, sucrose, sugar, xylitol, erythritol and mixtures thereof. In an embodiment, the composition of the invention is one wherein the pharmaceutically or cosmetically acceptable excipients or carriers comprises one or more filler; preferably comprises microcrystalline cellulose and its salts.

The term “disintegrant” refers to a substance which helps the composition break up once ingested. Materials commonly used as a disintegrant are, but not limited to, cross linked polyvinylpyrolidone; starches such as maize starch and dried sodium starch glycolate; gums such as maize starch and dried sodium starch glycolate; gums such as alginic acid, sodium alginate, guar gum; croscarmellose sodium; low-substituted hydroxypropyl cellulose and mixtures thereof. In an embodiment, the composition of the invention is one wherein the pharmaceutically or cosmetically acceptable excipients or carriers comprises one or more disintegrants; preferably comprises croscarmellose sodium.

The term “glidant” refers to a substance which improves the flow characteristics of powder mixtures in the dry state. Materials commonly used as a glidant include magnesium stearate, colloidal silicon dioxide or talc. In an embodiment, the composition of the invention is one wherein the pharmaceutically or cosmetically acceptable excipients or carriers comprises one or more glidant; preferably comprises magnesium stearate, talc or mixture thereof.

The term “lubricant” refers to a substance that prevents composition ingredients from clumping together and from sticking to the tablet punches or capsule filling machine and improves flowability of the composition mixture. Materials commonly used as a lubricant include sodium oleate, sodium stearate, sodium benzoate, sodium stearate, sodium chloride, stearic acid, sodium stearyl fumarate, calcium stearate, magnesium stearate, magnesium lauryl sulfate, sodium stearyl fumarate, sucrose esters or fatty acid, zinc, polyethylene glycol, talc and mixtures thereof. The presence of a lubricant is particularly preferred when the composition is a tablet to improve the tableting process. In an embodiment, the composition of the invention is one wherein the pharmaceutically or cosmetically acceptable excipients or carriers comprises one or more lubricants; preferably comprises magnesium stearate.

The pharmaceutical compositions of the present invention can be prepared according to methods well known in the state of the art. The appropriate excipients and/or carriers, and their amounts, can readily be determined by those skilled in the art according to the type of formulation being prepared.

### In Vitro Studies

### Idiopathic PD Cell Line

The use of human induced pluripotent stem cells (iPSCs) and iPSC-derived cells in this research was approved by the McGill University Research Ethics Board (IRB Study Number A03-M19-22A). Human iPSC line used in this experiment was a sporadic PD line QPN989. This line was generated from the PBMCs of a female patient with sporadic Parkinson's disease (PD). The age of collection for PBMCs was 42 years. Sequencing of common PD-associated genes (SNCA, PARK2, PARK6, GBA1, LRRK2, TMEM175, MAPT) revealed no mutations/variants and no genetic cause was attributed to the patient. IPSCs were reprogrammed from PBMCs with episomal plasmids. Once iPSC clones were obtained, they underwent a full quality control workflow as previously described, before being made available for use. Quality control included short tandem repeat (STR) profiling, genome stability testing, karyotyping, pluripotency checks and mycoplasma tests, in addition to trilineage tests. The QC process was described in an earlier study [99]. Dopaminergic neural progenitor cells (DA NPCs) generated from the sporadic PD line QPN989 were seeded in an automated manner into 96 well plates (15,000 cells seeded per well) in DA differentiation medium and allowed to attach for 24 h. The seeding of cells was automated using an EL-406 Washer Dispenser (BioTek) for plate coating, cell seeding, fixing, and staining as previously described in detail [100]. Once seeded, DNs were matured for 2 weeks in maintenance media as described previously [101]. Six replica plates were seeded per cell line. To avoid potential edge effects skewing the results, cells were only seeded into the inner 60 wells of each 96 well plate. After 2 weeks of differentiation, half of the medium was exchanged with fresh medium, and compounds/combos were added (3 single compounds and 4 combos: Creatine+CoQ10, TUDCA+CoQ10, TUDCA+Creatine and TUDCA+Creatine+CoQ10, also untreated PD control), using a contact free automated droplet dispenser (I.DOT, Dispendix). After 7 days, a half medium exchange and compound/combo addition was repeated. Four days later, this exchange/addition was repeated again. After 4 more days, dopaminergic neurons were fixed, stained, imaged and subsequently analyzed.

### Healthy Control Cell Line

Human iPSC control line used in this experiment was QPN929. This line was generated from the PBMCs of a healthy 50 year-old female. The reprogramming from PBMCs and seeding were as described above for the iPD cell line. We used 12 replicas per each sample tested.

After one week of differentiation, half of the medium was exchanged with fresh medium, and the individual testing compounds as well as TUDCA/CoQ10/Creatine combo and untreated control were added using a contact free droplet dispenser (I.DOT, Dispendix). From this point on, half medium exchange and compound/combo addition was repeated every 4 days. The incubation of the healthy control line was continued for an additional 2 weeks to a total of 4 week (total of 6 media/drug changes) before being fixed, stained, and imaged.

### Image and Data Statistical Analysis

Images were analyzed with Harmony image analysis software (Revvity). Before analysis, illumination in all images was corrected and image stacks were combined using maximum projection. Hoechst33342 stained nuclei were identified as objects to obtain the cell count per well. Neurofilament heavy (NFH) and beta-tubulin were evaluated in their respective channels by thresholding the image and filtering for cell bodies and filamentous structures (neurites). The identified objects were combined per image for each channel to calculate the total area occupied by neuronal cells per image. From the per image data the mean per well was calculated as final output. Further data processing was done in Excel (Microsoft), combining the replica plates to calculate the mean and standard deviation for each treatment. Each treatment result was normalized against the vehicle only control. Statistical significance was tested with two-sided, unpaired t-test.

### Human Microglia Cell Line

We evaluated the anti-neuroinflammatory effects of the combination of this invention by measuring effects on pro-inflammatory cytokines secretion following lipopolysaccharide (LPS) stimulation of human brain microglia cells. The selected doses were chosen based on the published literature and previous experience: CoQ10-10 μM; Creatine=10 μM; TUDCA=200 μM [33], [34], [31]. AIW002-2 Induced pluripotent stem cells (iPSCs) were obtained from a healthy donor as previously described and were differentiated into microglia following the described protocol [102]. Briefly, iPSCs were plated at DAY −1 onto Matrigel-coated 6-well plates aiming at a density of 1-5 small colonies. Differentiation into iPSC-derived hematopoietic progenitor cells (iHPCs) was carried out using STEMdiff™ Hematopoietic kit (STEMCELL Technologies). iHPC were collected at day 12 and 14 and plated onto Matrigel-coated vessel at a density of 5-10×10⁵ cells/mL in microglia differentiation medium 2.9 for 28 days. We followed the manufacturer's protocol for activation of the microglial cells with LPS to subsequently measure the effect of the study drugs on pro-inflammatory cytokines IL-6 and TNF-α. All cell culture procedures were performed in aseptic conditions, under a laminar flow hood. Microglia were thawed and cultured following the provider's instructions. The microglia were plated at 30,000 cells per well of a Corning Primaria 96 well plate (Ref N °353872) in 100 μL of growth medium. Cells were incubated at 37° C./5% CO₂ in a humidified cell culture incubator. Three days after cell plating, the test or reference substances (25 μL of solutions at 5 X) were applied 1 hour before LPS stimulation. One hour later, 100 ng/ml LPS was added to the cells and incubated for 16h at 37° C./5% CO₂ in a humidified cell culture incubator. After LPS incubation, the supernatant of each well was collected and centrifugated at 1000 g for 10 minutes to remove the cells and debris. Fifty microliters of supernatant from untreated and LPS-treated wells were used to determine cytokine secretion (IL-6 and TNF-α) with the Cytometric Bead Array Human Inflammatory Cytokines Kit (BD Falcon, cat. n. 551811) according to the provider's instructions. Readings were done on an Attune™ Nxt Flow Cytometer and analyzed/visualized using FlowJo™ software. Samples were diluted prior to the assay to fit within the standard range if needed. Six wells were used per each condition. Cytokines levels in the supernatant were expressed in pg/mL.

Statistical Analysis: Results were presented as means with the standard deviation (s.d.). One-way ANOVA followed by Dunnett's multiple comparisons test was used to analyse the data. All statistical calculations were performed using GraphPad Prism (Version 10.2.3).

### Results

### Effect of Experimental Conditions on Idiopathic PD and Healthy Control Cell Lines

The Parkinson's dopaminergic cell line treated with the combination of TUDCA/Creatine/CoQ10 for 14 days showed 24% increase in NFH filament area compared to the untreated cells (p<0.001) (Table 1 and FIG. 2A). Tubulin filament area was also increased by 16% (p<0.001) compared to the untreated cells (Table 2 and FIG. 2B). None of the three individual compounds alone or in other combinations showed an increase in these measures. We did not see any negative or positive changes in the cell number from the treatment with these supplements individually, or in any combinations (data not shown).

TABLE 1
NFH area, normalized to mean of control (plotted in FIG. 2A)

| | | | TUDCA/ | | | | | |
|---|---|---|---|---|---|---|---|---|
| | TUDCA/ | TUDCA/ | CoQ10/ | CoQ10/ | | | | |
| | CoQ10 | Creatine | Creatine | Creatine | TUDCA | CQ10 | Creatine | untreated |
| | 1.80* | 0.48* | 1.69 | 1.01 | 1.41 | 1.28 | 1.53 | 1.35 |
| | 1.28 | 1.30 | 1.47 | 1.04 | 1.18 | 1.24 | 1.17 | 1.38 |
| | 1.27 | 0.89 | 1.26 | 1.12 | 1.19 | 0.77 | 1.10 | 0.98 |
| | 1.09 | 1.05 | 1.13 | 0.88 | 1.10 | 0.94 | 1.03 | 0.86 |
| | 1.07 | 0.82 | 1.18 | 0.93 | 1.02 | 0.99 | 1.00 | 0.99 |
| | 0.93 | 0.87 | 1.07 | 1.06 | 0.98 | 1.12 | 0.97 | 0.93 |
| | 1.27 | 0.72 | 1.34 | 1.02 | 1.25 | 1.12 | 1.01 | 1.55 |
| | 0.99 | 0.90 | 1.17 | 0.95 | 1.04 | 0.98 | 1.03 | 1.13 |
| | 1.05 | 0.92 | 1.26 | 1.18 | 1.14 | 1.02 | 1.06 | 0.83 |
| | 0.95 | 0.90 | 1.00 | 1.00 | 0.78 | 0.99 | 0.90 | 1.19 |
| | 1.12 | 0.51* | 1.15 | 1.03 | 0.93 | 0.92 | 0.84 | 0.72 |
| | 0.98 | 0.95 | 1.16 | 0.97 | 0.72 | 0.91 | 0.80 | 0.57* |
| | | | | | | | | 1.41 |
| | | | | | | | | 1.16 |
| | | | | | | | | 0.86 |
| | | | | | | | | 0.94 |
| | | | | | | | | 0.88 |
| | | | | | | | | 0.74 |
| | | | | | | | | 1.51 |
| | | | | | | | | 1.11 |
| | | | | | | | | 0.85 |
| | | | | | | | | 1.00 |
| | | | | | | | | 0.77 |
| | | | | | | | | 0.76 |
| | | | | | | | | 1.82* |
| | | | | | | | | 1.10 |
| | | | | | | | | 0.85 |
| | | | | | | | | 0.66 |
| | | | | | | | | 0.78 |
| | | | | | | | | 0.78 |
| | | | | | | | | 1.94* |
| | | | | | | | | 0.49* |
| | | | | | | | | 0.76 |
| | | | | | | | | 0.98 |
| | | | | | | | | 1.07 |
| | | | | | | | | 0.75 |
| mean | 1.10 | 0.93 | 1.25 | 1.02 | 1.09 | 1.03 | 1.06 | 0.99 |
| stdv | 0.12 | 0.15 | 0.18 | 0.08 | 0.19 | 0.14 | 0.18 | 0.24 |

*Outliers removed

TABLE 2
Tubulin area, normalized to mean of control (plotted in FIG. 2B)

| | | | TUDCA/ | | | | | |
|---|---|---|---|---|---|---|---|---|
| | TUDCA/ | TUDCA/ | CoQ10/ | CoQ10/ | | | | |
| | CoQ10 | Creatine | Creatine | Creatine | TUDCA | CQ10 | Creatine | untreated |
| | 1.12 | 0.42* | 1.64* | 0.92 | 0.88 | 1.10 | 1.29* | 0.98 |
| | 1.12 | 0.42* | 1.30 | 0.92 | 0.88 | 1.10 | 0.96 | 1.02 |
| | 0.95 | 0.93 | 1.20 | 1.00 | 0.94 | 0.74 | 1.10 | 0.84 |
| | 1.01 | 1.00 | 1.14 | 0.87 | 1.15 | 0.98 | 1.04 | 0.80 |
| | 0.85 | 0.87 | 1.16 | 0.92 | 0.80 | 1.09 | 1.05 | 1.10 |
| | 0.99 | 0.90 | 1.00 | 1.02 | 1.12 | 1.10 | 1.02 | 1.26 |
| | 0.94 | 0.80 | 1.24 | 0.97 | 1.04 | 1.23 | 1.08 | 1.02 |
| | 0.99 | 0.88 | 1.10 | 0.90 | 1.18 | 1.02 | 1.11 | 0.85 |
| | 0.88 | 1.06 | 1.36 | 1.13 | 0.98 | 1.20 | 1.20 | 0.97 |
| | 1.03 | 0.97 | 1.05 | 0.96 | 0.96 | 1.10 | 1.04 | 1.13 |
| | 1.09 | 0.69* | 1.05 | 0.97 | 0.90 | 1.22 | 1.16 | 0.96 |
| | 1.19 | 1.10 | 1.17 | 0.90 | 0.98 | 1.13 | 1.04 | 0.94 |
| | | | | | | | | 1.04 |
| | | | | | | | | 0.96 |
| | | | | | | | | 0.91 |
| | | | | | | | | 0.98 |
| | | | | | | | | 0.99 |
| | | | | | | | | 1.12 |
| | | | | | | | | 1.08 |
| | | | | | | | | 1.37 |
| | | | | | | | | 1.08 |
| | | | | | | | | 1.01 |
| | | | | | | | | 0.88 |
| | | | | | | | | 1.03 |
| | | | | | | | | 1.12 |
| | | | | | | | | 0.88 |
| | | | | | | | | 0.99 |
| | | | | | | | | 0.92 |
| | | | | | | | | 1.11 |
| | | | | | | | | 1.22 |
| | | | | | | | | 0.78 |
| | | | | | | | | 0.77 |
| | | | | | | | | 0.87 |
| | | | | | | | | 1.06 |
| | | | | | | | | 1.29 |
| mean | 1.01 | 0.95 | 1.16 | 0.96 | 0.98 | 1.08 | 1.07 | 1.01 |
| stdv | 0.10 | 0.09 | 0.11 | 0.07 | 0.11 | 0.13 | 0.06 | 0.14 |

*Outliers removed

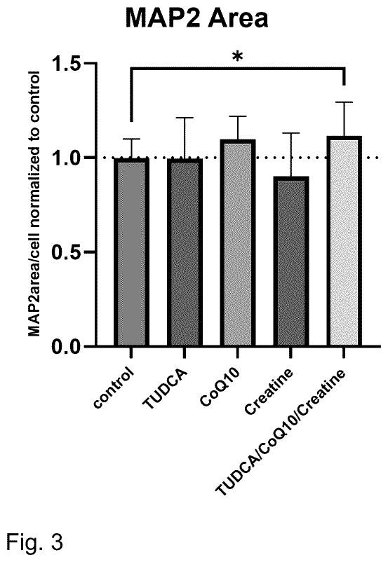

The healthy control cell line treated with the combination of TUDCA/Creatine/CoQ10 for 28 days showed no changes in NFH filament area compared to the untreated cells. However, the microtubule-associated protein 2 (MAP2) area was increased by 12% (p<0.05) compared to the untreated cells (Table 3 and FIG. 3). None of the three individual compounds alone produced a statistically significant increase in these measures. Tubulin filament was not tested. We did not see any negative or positive changes in the cell numbers from the treatment with these supplements individually, or in any combinations (data not shown).

TABLE 3
MAP2 area, normalized to mean of control (plotted in FIG. 3)

| | | TUDCA/ | | | |
|---|---|---|---|---|---|
| | | CoQ10/ | | | |
| | control | Creatine | TUDCA | CoQ10 | Creatine |
| | 0.93 | 1.22 | 1.17 | 1.18 | 1.03 |
| | 1.18 | 1.40 | 0.99 | 1.26 | 1.04 |
| | 0.96 | 1.10 | 0.99 | 1.04 | 0.98 |
| | 1.13 | 1.00 | 0.81 | 0.98 | 0.84 |
| | 0.97 | 1.00 | 1.21 | 1.04 | 0.86 |
| | 0.95 | 0.83 | 1.21 | 0.87 | 0.66 |
| | 1.07 | 1.12 | 0.49* | 1.00 | 0.35* |
| | 1.06 | 0.88 | 1.15 | 1.12 | 1.23 |
| | 0.77 | 1.13 | 0.77 | 1.20 | 0.78 |
| | 1.05 | 1.22 | 1.17 | 1.18 | 1.03 |
| | 0.88 | 1.40 | 0.99 | 1.26 | 1.04 |
| | 0.99 | 1.10 | 0.99 | 1.04 | 0.98 |
| | 1.17 | | | | |
| | 0.84 | | | | |
| | 0.99 | | | | |
| | 0.92 | | | | |
| | 1.06 | | | | |
| | 1.01 | | | | |
| | 0.45* | | | | |
| | 0.91 | | | | |
| | 1.35* | | | | |
| | 1.01 | | | | |
| | 0.96 | | | | |
| | 1.05 | | | | |
| | 1.06 | | | | |
| | 1.3* | | | | |
| | 0.93 | | | | |
| | 1.18 | | | | |
| | 0.96 | | | | |
| | 1.13 | | | | |
| | 0.97 | | | | |
| | 1.07 | | | | |
| | 1.06 | | | | |
| | 0.77 | | | | |
| mean | 1.00 | 1.12 | 1.04 | 1.10 | 0.95 |
| Std. | 0.10 | 0.18 | 0.15 | 0.12 | 0.16 |
| Dev. | | | | | |

*Outliers removed

### Effect of Experimental Conditions on Human Microglia Cell Line

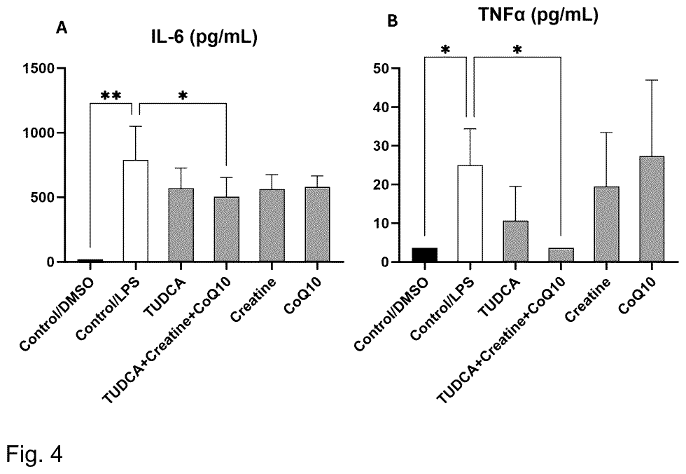

The combination of TUDCA/Creatine/CoQ10 reduced IL-6 levels demonstrating statistical significance. Whereas the individual supplements tested separately did not show statistically significant reduction of IL-6 even though such trend was seen (FIG. 4A). While TUDCA alone reduced another pro-inflammatory cytokine TNF-α, the triple combination of TUDCA/Creatine/CoQ10 completely abolished TNF-α even though neither creatine nor CoQ10 alone showed any statistically significant reduction in TNF-α, demonstrating a synergistic combined effect (FIG. 4B) of that combination.

TABLE 4
Effect of experimental conditions on IL-6 levels in activated
human microglial cells (plotted in FIG. 4A).

| | | | | TUDCA + | | |
|---|---|---|---|---|---|---|
| | Control/ | Control/ | | Creatine + | | |
| Value | DMSO | LPS | TUDCA | CoQ10 | Creatine | CoQ10 |
| Mean | 20 | 789.5 | 568.9 | 506 | 561.5 | 578.9 |
| Std. | | | | | | |
| Deviation | 0 | 261.6 | 156.3 | 147.5 | 113.8 | 87.09 |
| Std. Error | 0 | | 69.92 | | | |
| of Mean | | 117 | | 65.96 | 50.9 | 38.95 |
| Raw data | 20 | 629.679607 | 369.28641 | 322.804563 | 534.34439 | 525.09545 |
| | 20 | 623.987953 | 463.91017 | 418.139778 | 491.65698 | 534.34439 |
| | 20 | 1231.09778 | 750.62727 | 712.445751 | 762.95919 | 728.33495 |
| | 20 | 633.711196 | 574.18598 | 515.609363 | 517.50658 | 583.67207 |
| | 20 | 829.124671 | 686.59615 | 560.905447 | 500.90592 | 523.19824 |

TABLE 5
Effect of experimental conditions on TNF-α levels
in activated human microglial cells (Plotted in FIG. 4B)

| | | | | TUDCA + | | |
|---|---|---|---|---|---|---|
| | Control/ | Control/ | | Creatine + | | |
| Value | DMSO | LPS | TUDCA | CoQ10 | Creatine | CoQ10 |
| Mean | 3.7 | 25 | 10.65 | 3.7 | 20.23 | 27.37 |
| Std. | | | | | | |
| Deviation | 0 | 9.409 | 8.872 | 0 | 12.72 | 19.59 |
| Std. Error of | 0 | | | 0 | 5.689 | 8.762 |
| Mean | | 4.208 | 3.968 | | | |
| Raw values | 3.7 | 25.6616201 | 10.2613311 | 3.7 | 27.32011 | 49.5913 |
| | 3.7 | 29.215533 | 24.0031274 | 3.7 | 25.66162 | 15.47374 |
| | 3.7 | 35.8495036 | 13.5783164 | 3.7 | 34.19101 | 47.93281 |
| | 3.7 | 10.2613311 | 3.7* | 3.7 | 10.26133 | 11.91982 |
| | 3.7 | 24.0031274 | 17.319402 | 3.7 | 3.7* | 11.91982 |

*Outliers removed

### Discussion

This work identified a promising combination of the three dietary supplements TUDCA or UDCA, CoQ10 as ubiquinone or ubiquinol, and creatine described above for further preclinical and clinical validation. Indeed, the three tested agents together are thought to affect most of the known pathways seen in neurodegeneration such as aggregation of misfolded proteins, neuroinflammation, mitochondrial dysfunction, ROS formation, and iron accumulation leading to neurodegeneration (FIG. 1). This triple combination improved NFH and tubulin levels in dopaminergic neurons from PD suggesting, if not neuroprotection, but at least enhancement of well-being of these cells.

Interestingly, the triple combination resulted in increased MAP2 level in healthy control dopamine neurons. MAP2 is involved in interactions with cytoskeletal filaments, process formation, synaptic plasticity, and regulation of protein folding and transport. Neuron loss, regardless of cause, is accompanied by corresponding loss of MAP2 [103]. Therefore, these supplements as a combination should be further evaluated for possible prevention of NDs and other age-related conditions.

Anti-inflammatory effects of the individual drugs were observed with CoQ10 at 10 μM, TUDCA at 200 UM and creatine at 10μM. These effects were potentiated when the treatments were combined. The most impressive results were seen with a triple combination of CoQ10, TUDCA and creatine which statistically significantly decreased levels of the measured cytokines, especially TNF-α suggesting reduction of neuroinflammation, (FIG. 4). Therefore, this combination might potentially be used to treat or delay onset of many NDs described above since neuroinflammation is a hallmark of all of them.

Peripheral inflammation might contribute to neuroinflammation via alterations in blood brain barrier and propagation in the CNS by direct neuronal routes. Once inflammatory signals are received by microglia, it gets activated and maintains neuroinflammatory state. Analyses of human brain tissues on autopsies of RA patients revealed microglial activation [9].

TNF-α is a central regulator of inflammation in the human body. It is also involved in the pathogenesis of various peripheral autoimmune and inflammatory diseases, including rheumatoid arthritis (RA), and inflammatory bowel diseases (IBD) such as Crohn's disease (CD) and ulcerative colitis (UC). In fact, TNF-α inhibitors, including etanercept (E), infliximab (I), adalimumab (A), certolizumab pegol (C), and golimumab (G), are biologic agents which are FDA-approved to treat such autoimmune disorders as: ankylosing spondylitis (E, I, A, C, and G), Crohn disease (I, A and C), hidradenitis suppurativa (A), juvenile idiopathic arthritis (A), plaque psoriasis (E, I and A), polyarticular juvenile idiopathic arthritis (E), psoriatic arthritis (E, I, A, C, and G), rheumatoid arthritis (E, I, A, C, and G), ulcerative colitis (I, A and G), and uveitis (A). TNF-α is also highly elevated in inclusion body myositis, a neurodegenerative myopathy characterized by accumulation of misfolded proteins, mitochondrial dysfunction and inflammation [18]. Therefore, the triple combination of TUDCA, creatine and CoQ10 is expected to be useful for treatment of these autoimmune and inflammatory conditions as well, since it showed dramatic reduction in TFN-α in human activated microglia.

Overall, we described a preclinical screening and design of drug combinations based on their different mechanisms of action that together could lead to development of a product capable of providing clinically significant neuroprotection in a major ND. The integration of these three compounds into therapeutic strategies offers hope for improved treatments and outcomes for patients suffering from neurodegenerative and autoimmune diseases.

The collective findings on the combination of TUDCA, CoQ10 and Creatine highlight their unexpected synergistic therapeutic potential in neurodegenerative and possibly autoimmune diseases. Their diverse mechanisms of action address various aspects of ND pathology, specifically oxidative stress, mitochondrial dysfunction and neuroinflammation. Given the conceptual similarities in pathological pathways occurring in the neurodegenerative diseases described above and the provided evidence from the literature showing that the three supplements individually exhibit neuroprotective effect in different preclinical models of PD, AD, ALS and HD we believe that the triple combination of TUDCA, CoQ10 and Creatine should also show superior efficacy not only in PD, but in all the above diseases. To prove this, we chose an in-vitro model of Parkinson's Disease as an example to illustrate advantage of combining these supplements to achieve better efficacy. Therefore, results of these experiments can be extrapolated to the other ND's described here. Unexpected positive effect of the triple combination on neuronal biomarker MAP2 in healthy dopaminergic neurons is encouraging as well making this combination of supplements potentially useful in prevention of NDs.

The noted anti-inflammatory effect manifested by unexpected, marked reduction of TNF-α, could also make the described supplement combination useful for treatment of autoimmune diseases that are known to improve by inhibition of TNF-α.

### REFERENCES

ADDIN EN.REFLIST (1) Dugger, B. N.; Dickson, D. W. Pathology of Neurodegenerative Diseases. Cold Spring Harb Perspect Biol 2017, 9 (7). DOI: 10.1101/cshperspect.a028035.
(2) Ahmed, R. M.; Devenney, E. M.; Irish, M.; Ittner, A.; Naismith, S.; Ittner, L. M.; Rohrer, J. D.; Halliday, G. M.; Eisen, A.; Hodges, J. R.; et al. Neuronal network disintegration: common pathways linking neurodegenerative diseases. J Neurol Neurosurg Psychiatry 2016, 87 (11), 1234-1241. DOI: 10.1136/jnnp-2014-308350.
(3) Wang, J.; Gu, B. J.; Masters, C. L.; Wang, Y. J. A systemic view of Alzheimer disease—insights from amyloid-beta metabolism beyond the brain. Nat Rev Neurol 2017, 13 (11), 703. DOI: 10.1038/nrneurol.2017.147.
(4) Zong, J.; Yang, Y.; Wang, H.; Zhang, H.; Yang, X.; Yang, X. The two-directional prospective association between inflammatory bowel disease and neurodegenerative disorders: a systematic review and meta-analysis based on longitudinal studies. Front Immunol 2024, 15, 1325908. DOI: 10.3389/fimmu.2024.1325908 From NLM Medline.
(5) Wallin, K.; Solomon, A.; Kareholt, I.; Tuomilehto, J.; Soininen, H.; Kivipelto, M. Midlife rheumatoid arthritis increases the risk of cognitive impairment two decades later: a population-based study. J Alzheimers Dis 2012, 31 (3), 669-676. DOI: 10.3233/JAD-2012-111736 From NLM Medline.
(6) Abbott, R.; Whear, R.; Nikolaou, V.; Bethel, A.; Coon, J. T.; Stein, K.; Dickens, C. Tumour necrosis factor-alpha inhibitor therapy in chronic physical illness: A systematic review and meta-analysis of the effect on depression and anxiety. J Psychosom Res 2015, 79 (3), 175-184. DOI: 10.1016/j.jpsychores.2015.04.008 From NLM Medline.
(7) Kappelmann, N.; Lewis, G.; Dantzer, R.; Jones, P. B.; Khandaker, G. M. Antidepressant activity of anti-cytokine treatment: a systematic review and meta-analysis of clinical trials of chronic inflammatory conditions. Mol Psychiatry 2018, 23 (2), 335-343. DOI: 10.1038/mp.2016.167 From NLM Medline.
(8) Chou, R. C.; Kane, M.; Ghimire, S.; Gautam, S.; Gui, J. Treatment for Rheumatoid Arthritis and Risk of Alzheimer's Disease: A Nested Case-Control Analysis. CNS Drugs 2016, 30 (11), 1111-1120. DOI: 10.1007/s40263-016-0374-z From NLM Medline.
(9) Suss, P.; Rothe, T.; Hoffmann, A.; Schlachetzki, J. C. M.; Winkler, J. The Joint-Brain Axis: Insights From Rheumatoid Arthritis on the Crosstalk Between Chronic Peripheral Inflammation and the Brain. Front Immunol 2020, 11, 612104. DOI: 10.3389/fimmu.2020.612104 From NLM Medline.
(10) Cuervo, A. M.; Stefanis, L.; Fredenburg, R.; Lansbury, P. T.; Sulzer, D. Impaired degradation of mutant alpha-synuclein by chaperone-mediated autophagy. Science 2004, 305 (5688), 1292-1295. DOI: 10.1126/science.1101738.
(11) Mosharov, E. V.; Larsen, K. E.; Kanter, E.; Phillips, K. A.; Wilson, K.; Schmitz, Y.; Krantz, D. E.; Kobayashi, K.; Edwards, R. H.; Sulzer, D. Interplay between cytosolic dopamine, calcium, and alpha-synuclein causes selective death of substantia nigra neurons. Neuron 2009, 62 (2), 218-229. DOI: 10.1016/j.neuron.2009.01.033.
(12) Ryan, S. D.; Dolatabadi, N.; Chan, S. F.; Zhang, X.; Akhtar, M. W.; Parker, J.; Soldner, F.; Sunico, C. R.; Nagar, S.; Talantova, M.; et al. Isogenic human iPSC Parkinson's model shows nitrosative stress-induced dysfunction in MEF2-PGC1alpha transcription. Cell 2013, 155 (6), 1351-1364. DOI: 10.1016/j.cell.2013.11.009.
(13) Zucca, F. A.; Segura-Aguilar, J.; Ferrari, E.; Munoz, P.; Paris, I.; Sulzer, D.; Sarna, T.; Casella, L.; Zecca, L. Interactions of iron, dopamine and neuromelanin pathways in brain aging and Parkinson's disease. Prog Neurobiol 2017, 155, 96-119. DOI: 10.1016/j.pneurobio.2015.09.012.
(14) Hurley, M. J.; Brandon, B.; Gentleman, S. M.; Dexter, D. T. Parkinson's disease is associated with altered expression of CaV1 channels and calcium-binding proteins. Brain 2013, 136 (Pt 7), 2077-2097. DOI: 10.1093/brain/awt134.
(15) Rocha, E. M.; De Miranda, B.; Sanders, L. H. Alpha-synuclein: Pathology, mitochondrial dysfunction and neuroinflammation in Parkinson's disease. Neurobiol Dis 2018, 109 (Pt B), 249-257. DOI: 10.1016/j.nbd.2017.04.004.
(16) Vijiaratnam, N.; Simuni, T.; Bandmann, O.; Morris, H. R.; Foltynie, T. Progress towards therapies for disease modification in Parkinson's disease. Lancet Neurol 2021, 20 (7), 559-572. DOI: 10.1016/S1474-4422 (21) 00061-2.
(17) Lang, A. E.; Espay, A. J. Disease Modification in Parkinson's Disease: Current Approaches, Challenges, and Future Considerations. Mov Disord 2018, 33 (5), 660-677. DOI: 10.1002/mds.27360.
(18) Keller, C. W.; Schmidt, J.; Lunemann, J. D. Immune and myodegenerative pathomechanisms in inclusion body myositis. Ann Clin Transl Neurol 2017, 4 (6), 422-445. DOI: 10.1002/acn3.419.
(19) Matthews, R. T.; Yang, L.; Browne, S.; Baik, M.; Beal, M. F. Coenzyme Q10 administration increases brain mitochondrial concentrations and exerts neuroprotective effects. Proc Natl Acad Sci U S A 1998, 95 (15), 8892-8897. DOI: 10.1073/pnas.95.15.8892 From NLM Medline.
(20) Ohtsuki, S.; Tachikawa, M.; Takanaga, H.; Shimizu, H.; Watanabe, M.; Hosoya, K.; Terasaki, T. The blood-brain barrier creatine transporter is a major pathway for supplying creatine to the brain. J Cereb Blood Flow Metab 2002, 22 (11), 1327-1335. DOI: 10.1097/01.WCB.0000033966.83623.7D From NLM Medline.
(21) Parry, G. J.; Rodrigues, C. M.; Aranha, M. M.; Hilbert, S. J.; Davey, C.; Kelkar, P.; Low, W. C.; Steer, C. J. Safety, tolerability, and cerebrospinal fluid penetration of ursodeoxycholic Acid in patients with amyotrophic lateral sclerosis. Clin Neuropharmacol 2010, 33 (1), 17-21. DOI: 10.1097/WNF.0b013e3181c47569 From NLM Medline.
(22) Verma, M.; Lizama, B. N.; Chu, C. T. Excitotoxicity, calcium and mitochondria: a triad in synaptic neurodegeneration. Transl Neurodegener 2022, 11 (1), 3. DOI: 10.1186/s40035-021-00278-7 From NLM.
(23) Pham, C. G.; Bubici, C.; Zazzeroni, F.; Papa, S.; Jones, J.; Alvarez, K.; Jayawardena, S.; De Smaele, E.; Cong, R.; Beaumont, C.; et al. Ferritin Heavy Chain Upregulation by NF-κB Inhibits TNFα-Induced Apoptosis by Suppressing Reactive Oxygen Species. Cell 2004, 119 (4), 529-542. DOI: 10.1016/j.cell.2004.10.017.
(24) Dias, V.; Junn, E.; Mouradian, M. M. The role of oxidative stress in Parkinson's disease. Journal of Parkinson's disease 2013, 3 (4), 461-491. DOI: 10.3233/JPD-130230 PubMed.
(25) Haining, R.; Achat-Mendes, C. Neuromelanin, one of the most overlooked molecules in modern medicine, is not a spectator. Neural Regeneration Research 2017, 12 (3), 372. DOI: 10.4103/1673-5374.202928.
(26) Gerlach, M.; Double, K. L.; Ben-Shachar, D.; Zecca, L.; Youdim, M. B. H.; Riederer, P. Neuromelanin and its interaction with iron as a potential risk factor for dopaminergic neurodegeneration underlying Parkinson's disease. Neurotoxicity Research 2003, 5 (1-2), 35-43. DOI: 10.1007/bf03033371.
(27) Viceconte, N.; Burguillos, M. A.; Herrera, A. J.; De Pablos, R. M.; Joseph, B.; Venero, J. L. Neuromelanin activates proinflammatory microglia through a caspase-8-dependent mechanism. Journal of Neuroinflammation 2015, 12 (1), 5. DOI: 10.1186/s12974-014-0228-x.
(28) Cortez, L.; Sim, V. The therapeutic potential of chemical chaperones in protein folding diseases. Prion 2014, 8 (2). DOI: 10.4161/pri.28938.
(29) Vang, S.; Longley, K.; Steer, C. J.; Low, W. C. The Unexpected Uses of Urso-and Tauroursodeoxycholic Acid in the Treatment of Non-liver Diseases. Glob Adv Health Med 2014, 3 (3), 58-69. DOI: 10.7453/gahmj.2014.017.
(30) Soto, C.; Pritzkow, S. Protein misfolding, aggregation, and conformational strains in neurodegenerative diseases. Nat Neurosci 2018, 21 (10), 1332-1340. DOI: 10.1038/s41593-018-0235-9.
(31) Kim, S. J.; Ko, W. K.; Jo, M. J.; Arai, Y.; Choi, H.; Kumar, H.; Han, I. B.; Sohn, S. Anti-inflammatory effect of Tauroursodeoxycholic acid in RAW 264.7 macrophages, Bone marrow-derived macrophages, BV2 microglial cells, and spinal cord injury. Sci Rep 2018, 8 (1), 3176. DOI: 10.1038/s41598-018-21621-5 From NLM Medline.
(32) Mendes, M. O.; Rosa, A. I.; Carvalho, A. N.; Nunes, M. J.; Dionisio, P.; Rodrigues, E.; Costa, D.; Duarte-Silva, S.; Maciel, P.; Rodrigues, C. M. P.; et al. Neurotoxic effects of MPTP on mouse cerebral cortex: Modulation of neuroinflammation as a neuroprotective strategy. Mol Cell Neurosci 2019, 96, 1-9. DOI: 10.1016/j.mcn.2019.01.003 From NLM Medline.
(33) Romero-Ramirez, L.; Garcia-Rama, C.; Wu, S.; Mey, J. Bile acids attenuate PKM2 pathway activation in proinflammatory microglia. Sci Rep 2022, 12 (1), 1459. DOI: 10.1038/s41598-022-05408-3 From NLM Medline.
(34) Yanguas-Casas, N.; Barreda-Manso, M. A.; Nieto-Sampedro, M.; Romero-Ramirez, L. TUDCA: An Agonist of the Bile Acid Receptor GPBAR1/TGR5 With Anti-Inflammatory Effects in Microglial Cells. J Cell Physiol 2017, 232 (8), 2231-2245. DOI: 10.1002/jcp.25742 From NLM Medline.
(35) Cortez, L. M.; Campeau, J.; Norman, G.; Kalayil, M.; Van der Merwe, J.; Mckenzie, D.; Sim, V. L. Bile Acids Reduce Prion Conversion, Reduce Neuronal Loss, and Prolong Male Survival in Models of Prion Disease. J Virol 2015, 89 (15), 7660-7672. DOI: 10.1128/JVI.01165-15.
(36) Lo, A. C.; Callaerts-Vegh, Z.; Nunes, A. F.; Rodrigues, C. M.; D'Hooge, R. Tauroursodeoxycholic acid (TUDCA) supplementation prevents cognitive impairment and amyloid deposition in APP/PS1 mice. Neurobiol Dis 2013, 50, 21-29. DOI: 10.1016/j.nbd.2012.09.003.
(37) Nunes, A. F.; Amaral, J. D.; Lo, A. C.; Fonseca, M. B.; Viana, R. J.; Callaerts-Vegh, Z.; D'Hooge, R.; Rodrigues, C. M. TUDCA, a bile acid, attenuates amyloid precursor protein processing and amyloid-beta deposition in APP/PSI mice. Mol Neurobiol 2012, 45 (3), 440-454. DOI: 10.1007/s12035-012-8256-y.
(38) Kusaczuk, M. Tauroursodeoxycholate-Bile Acid with Chaperoning Activity: Molecular and Cellular Effects and Therapeutic Perspectives. Cells 2019, 8 (12). DOI: 10.3390/cells8121471.
(39) Sola, S.; Castro, R. E.; Laires, P. A.; Steer, C. J.; Rodrigues, C. M. Tauroursodeoxycholic acid prevents amyloid-beta peptide-induced neuronal death via a phosphatidylinositol 3-kinase-dependent signaling pathway.Mol Med 2003, 9 (9-12), 226-234. DOI: 10.2119/2003-00042.rodrigues.
(40) Duan, W. M.; Rodrigues, C. M.; Zhao, L. R.; Steer, C. J.; Low, W. C. Tauroursodeoxycholic acid improves the survival and function of nigral transplants in a rat model of Parkinson's disease. Cell Transplant 2002, 11 (3), 195-205.
(41) Weihe, E.; Depboylu, C.; Schutz, B.; Schafer, M. K.; Eiden, L. E. Three types of tyrosine hydroxylase-positive CNS neurons distinguished by dopa decarboxylase and VMAT2 co-expression. Cell Mol Neurobiol 2006, 26 (4-6), 659-678. DOI: 10.1007/s10571-006-9053-9.
(42) Castro-Caldas, M.; Carvalho, A. N.; Rodrigues, E.; Henderson, C. J.; Wolf, C. R.; Rodrigues, C. M.; Gama, M. J. Tauroursodeoxycholic acid prevents MPTP-induced dopaminergic cell death in a mouse model of Parkinson's disease. Mol Neurobiol 2012, 46 (2), 475-486. DOI: 10.1007/s12035-012-8295-4.
(43) Cuevas, E.; Burks, S.; Raymick, J.; Robinson, B.; Gomez-Crisostomo, N. P.; Escudero-Lourdes, C.; Lopez, A. G. G.; Chigurupati, S.; Hanig, J.; Ferguson, S. A.; et al. Tauroursodeoxycholic acid (TUDCA) is neuroprotective in a chronic mouse model of Parkinson's disease. Nutr Neurosci 2020, 1-18. DOI: 10.1080/1028415X.2020.1859729.
(44) Rosa, A. I.; Fonseca, I.; Nunes, M. J.; Moreira, S.; Rodrigues, E.; Carvalho, A. N.; Rodrigues, C. M. P.; Gama, M. J.; Castro-Caldas, M. Novel insights into the antioxidant role of tauroursodeoxycholic acid in experimental models of Parkinson's disease. Biochim Biophys Acta Mol Basis Dis 2017, 1863 (9), 2171-2181. DOI: 10.1016/j.bbadis.2017.06.004.
(45) Elia, A. E.; Lalli, S.; Monsurro, M. R.; Sagnelli, A.; Taiello, A. C.; Reggiori, B.; La Bella, V.; Tedeschi, G.; Albanese, A. Tauroursodeoxycholic acid in the treatment of patients with amyotrophic lateral sclerosis. Eur J Neurol 2016, 23 (1), 45-52. DOI: 10.1111/ene.12664.
(46) Cohen, J. B.; Klee, J. COMPOSITIONS FOR IMPROVING CELL VIABILITY AND METHODS OF USE THEREOF. WO 2014/158547 A1, 2014.
(47) Keene, C. D.; Rodrigues, C. M.; Eich, T.; Linehan-Stieers, C.; Abt, A.; Kren, B. T.; Steer, C. J.; Low, W. C. A bile acid protects against motor and cognitive deficits and reduces striatal degeneration in the 3-nitropropionic acid model of Huntington's disease. Exp Neurol 2001, 171 (2), 351-360. DOI: 10.1006/exnr.2001.7755.
(48) Keene, C. D.; Rodrigues, C. M.; Eich, T.; Chhabra, M. S.; Steer, C. J.; Low, W. C. Tauroursodeoxycholic acid, a bile acid, is neuroprotective in a transgenic animal model of Huntington's disease. Proc Natl Acad Sci U S A 2002, 99 (16), 10671-10676. DOI: 10.1073/pnas. 162362299.
(49) Van den Bossche, L.; Borsboom, D.; Devriese, S.; Van Welden, S.; Holvoet, T.; Devisscher, L.; Hindryckx, P.; De Vos, M.; Laukens, D. Tauroursodeoxycholic acid protects bile acid homeostasis under inflammatory conditions and dampens Crohn's disease-like ileitis. Laboratory Investigation 2017, 97 (5), 519-529.
(50) Yang, Y.; He, J.; Suo, Y.; Zheng, Z.; Wang, J.; Lv, L.; Huo, C.; Wang, Z.; Li, J.; Sun, W. Tauroursodeoxycholate improves 2, 4, 6-trinitrobenzenesulfonic acid-induced experimental acute ulcerative colitis in mice. International Immunopharmacology 2016, 36, 271-276.
(51) Zhao, J.; Hao, S.; Chen, Y.; Ye, X.; Fang, P.; Hu, H. Tauroursodeoxycholic acid liposome alleviates DSS-induced ulcerative colitis through restoring intestinal barrier and gut microbiota. Colloids and Surfaces B: Biointerfaces 2024, 113798.
(52) Lee, D.; Shim, M. S.; Kim, K. Y.; Noh, Y. H.; Kim, H.; Kim, S. Y.; Weinreb, R. N.; Ju, W. K. Coenzyme Q10 inhibits glutamate excitotoxicity and oxidative stress-mediated mitochondrial alteration in a mouse model of glaucoma. Invest Ophthalmol Vis Sci 2014, 55 (2), 993-1005. DOI: 10.1167/iovs.13-12564 From NLM Medline.
(53) Rauchova, H. Coenzyme Q10 effects in neurological diseases. Physiological Research 2021, 70 (Suppl 4), S683.
(54) Yang, X.; Dai, G.; Li, G.; Yang, E. S. Coenzyme Q10 reduces β-amyloid plaque in an APP/PS1 transgenic mouse model of Alzheimer's disease. Journal of molecular neuroscience 2010, 41, 110-113.
(55) Dumont, M.; Kipiani, K.; Yu, F.; Wille, E.; Katz, M.; Calingasan, N. Y.; Gouras, G. K.; Lin, M. T.; Beal, M. F. Coenzyme Q10 decreases amyloid pathology and improves behavior in a transgenic mouse model of Alzheimer's disease. Journal of Alzheimer's disease 2011, 27 (1), 211-223.
(56) Komaki, H.; Faraji, N.; Komaki, A.; Shahidi, S.; Etaee, F.; Raoufi, S.; Mirzaei, F. Investigation of protective effects of coenzyme Q10 on impaired synaptic plasticity in a male rat model of Alzheimer's disease. Brain research bulletin 2019, 147, 14-21.
(57) Ishrat, T.; Khan, M. B.; Hoda, M. N.; Yousuf, S.; Ahmad, M.; Ansari, M. A.; Ahmad, A. S.; Islam, F. Coenzyme Q10 modulates cognitive impairment against intracerebroventricular injection of streptozotocin in rats. Behavioural brain research 2006, 171 (1), 9-16.
(58) Muthukumaran, K.; Kanwar, A.; Vegh, C.; Marginean, A.; Elliott, A.; Guilbeault, N.; Badour, A.; Sikorska, M.; Cohen, J.; Pandey, S. Ubisol-Q 10 (a nanomicellar water-soluble formulation of CoQ 10) treatment inhibits Alzheimer-type behavioral and pathological symptoms in a double transgenic mouse (TgAPEswe, PSEN1dE9) model of Alzheimer's disease. Journal of Alzheimer's Disease 2018, 61 (1), 221-236.
(59) Wear, D.; Vegh, C.; Sandhu, J. K.; Sikorska, M.; Cohen, J.; Pandey, S. Ubisol-Q10, a Nanomicellar and Water-Dispersible Formulation of Coenzyme-Q10 as a Potential Treatment for Alzheimer's and Parkinson's Disease. Antioxidants 2021, 10 (5), 764.
(60) Beal, M. F. Mitochondrial dysfunction and oxidative damage in Alzheimer's and Parkinson's diseases and coenzyme Q 10 as a potential treatment. Journal of bioenergetics and biomembranes 2004, 36, 381-386.
(61) Shults, C. W.; Oakes, D.; Kieburtz, K.; Beal, M. F.; Haas, R.; Plumb, S.; Juncos, J. L.; Nutt, J.; Shoulson, I.; Carter, J. Effects of coenzyme Q10 in early Parkinson disease: evidence of slowing of the functional decline. Archives of neurology 2002, 59 (10), 1541-1550.
(62) Cleren, C.; Yang, L.; Lorenzo, B.; Calingasan, N. Y.; Schomer, A.; Sireci, A.; Wille, E. J.; Beal, M. F. Therapeutic effects of coenzyme Q10 (CoQ10) and reduced CoQ10 in the MPTP model of Parkinsonism. Journal of neurochemistry 2008, 104 (6), 1613-1621.
(63) Somayajulu-Niţu, M.; Sandhu, J. K.; Cohen, J.; Sikorska, M.; Sridhar, T.; Matei, A.; Borowy-Borowski, H.; Pandey, S. Paraquat induces oxidative stress, neuronal loss in substantia nigra region and Parkinsonism in adult rats: neuroprotection and amelioration of symptoms by water-soluble formulation of coenzyme Q 10. BMC neuroscience 2009, 10, 1-12.
(64) Attia, H. N.; Maklad, Y. A. Neuroprotective effects of coenzyme Q10 on paraquat-induced Parkinson's disease in experimental animals. Behavioural pharmacology 2018, 29 (1), 79-86.
(65) Kooncumchoo, P.; Sharma, S.; Porter, J.; Govitrapong, P.; Ebadi, M. Coenzyme Q (10) provides neuroprotection in iron-induced apoptosis in dopaminergic neurons. J Mol Neurosci 2006, 28 (2), 125-141. DOI: 10.1385/JMN: 28:2:125 From NLM Medline.
(66) Park, H. W.; Park, C. G.; Park, M.; Lee, S. H.; Park, H. R.; Lim, J.; Pack, S. H.; Choy, Y. B. Intrastriatal administration of coenzyme Q10 enhances neuroprotection in a Parkinson's disease rat model. Scientific Reports 2020, 10 (1), 9572.
(67) Parkinson Study Group, Q. E. I.; Beal, M. F.; Oakes, D.; Shoulson, I.; Henchcliffe, C.; Galpern, W. R.; Haas, R.; Juncos, J. L.; Nutt, J. G.; Voss, T. S.; et al. A randomized clinical trial of high-dosage coenzyme Q10 in early Parkinson disease: no evidence of benefit. JAMA Neurol 2014, 71 (5), 543-552. DOI: 10.1001/jamaneurol.2014.131.
(68) Yang, L.; Calingasan, N. Y.; Wille, E. J.; Cormier, K.; Smith, K.; Ferrante, R. J.; Flint Beal, M. Combination therapy with coenzyme Q10 and creatine produces additive neuroprotective effects in models of Parkinson's and Huntington's diseases. Journal of neurochemistry 2009, 109 (5), 1427-1439.
(69) Hickey, M. A.; Zhu, C.; Medvedeva, V.; Franich, N. R.; Levine, M. S.; Chesselet, M.-F. Evidence for behavioral benefits of early dietary supplementation with CoEnzymeQ10 in a slowly progressing mouse model of Huntington's disease. Molecular and Cellular Neuroscience 2012, 49 (2), 149-157.
(70) Koroshetz, W. J.; Jenkins, B. G.; Rosen, B. R.; Beal, M. F. Energy metabolism defects in Huntington's disease and effects of coenzyme Q10. Annals of Neurology: Official Journal of the American Neurological Association and the Child Neurology Society 1997, 41 (2), 160-165.
(71) Ferrante, R. J.; Andreassen, O. A.; Dedeoglu, A.; Ferrante, K. L.; Jenkins, B. G.; Hersch, S. M.; Beal, M. F. Therapeutic effects of coenzyme Q10 and remacemide in transgenic mouse models of Huntington's disease. Journal of Neuroscience 2002, 22 (5), 1592-1599.
(72) Schilling, G.; Coonfield, M. L.; Ross, C. A.; Borchelt, D. R. Coenzyme Q10 and remacemide hydrochloride ameliorate motor deficits in a Huntington's disease transgenic mouse model. Neuroscience letters 2001, 315 (3), 149-153.
(73) Abdollahzad, H.; Aghdashi, M. A.; Jafarabadi, M. A.; Alipour, B. Effects of coenzyme Q10 supplementation on inflammatory cytokines (TNF-α, IL-6) and oxidative stress in rheumatoid arthritis patients: a randomized controlled trial. Archives of medical research 2015, 46 (7), 527-533.
(74) Nachvak, S. M.; Alipour, B.; Mahdavi, A. M.; Aghdashi, M. A.; Abdollahzad, H.; Pasdar, Y.; Samadi, M.; Mostafai, R. Effects of coenzyme Q10 supplementation on matrix metalloproteinases and DAS-28 in patients with rheumatoid arthritis: a randomized, double-blind, placebo-controlled clinical trial. Clinical Rheumatology 2019, 38, 3367-3374.
(75) Farsi, F.; Ebrahimi-Daryani, N.; Barati, M.; Janani, L.; Karimi, M. Y.; Akbari, A.; Irandoost, P.; Alamdari, N. M.; Agah, S.; Vafa, M. Effects of coenzyme Q10 on health-related quality of life, clinical disease activity and blood pressure in patients with mild to moderate ulcerative colitis: a randomized clinical trial. Medical Journal of the Islamic Republic of Iran 2021, 35, 3.
(76) El Morsy, E. M.; Kamel, R.; Ahmed, M. A. Attenuating effects of coenzyme Q10 and amlodipine in ulcerative colitis model in rats. Immunopharmacology and immunotoxicology 2015, 37 (3), 244-251.
(77) Ewees, M. G.; Messiha, B. A. S.; Abo-Saif, A. A.; Abd El, H. A. E.-T. Is coenzyme Q10 effective in protection against ulcerative colitis? An experimental study in rats. Biological and Pharmaceutical Bulletin 2016, 39 (7), 1159-1166.
(78) Al-Oudah, G. A.; Sahib, A. S.; Al-Hattab, M. K.; Al-Ameedee, A. A. Effect of CoQ10 Administration to Psoriatic Iraqi Patients on Biological Therapy Upon Severity Index (PASI) and Quality of Life Index (DLQI) Before and After Therapy. Journal of Population Therapeutics and Clinical Pharmacology 2022, 29 (02).
(79) Roschel, H.; Gualano, B.; Ostojic, S. M.; Rawson, E. S. Creatine supplementation and brain health. Nutrients 2021, 13 (2), 586.
(80) Snow, W. M.; Cadonic, C.; Cortes-Perez, C.; Adlimoghaddam, A.; Roy Chowdhury, S. K.; Thomson, E.; Anozie, A.; Bernstein, M. J.; Gough, K.; Fernyhough, P. Sex-specific effects of chronic creatine supplementation on hippocampal-mediated spatial cognition in the 3xTg mouse model of Alzheimer's disease. Nutrients 2020, 12 (11), 3589.
(81) Brewer, G. J.; Wallimann, T. W. Protective effect of the energy precursor creatine against toxicity of glutamate and β-amyloid in rat hippocampal neurons. Journal of neurochemistry 2000, 74 (5), 1968-1978.
(82) Smith, A. N.; Morris, J. K.; Carbuhn, A. F.; Herda, T. J.; Keller, J. E.; Sullivan, D. K.; Taylor, M. K. Creatine as a therapeutic target in Alzheimer's disease. Current Developments in Nutrition 2023, 102011.
(83) Genius, J.; Geiger, J.; Bender, A.; Moller, H. J.; Klopstock, T.; Rujescu, D. Creatine protects against excitoxicity in an in vitro model of neurodegeneration. PLOS One 2012, 7 (2), e30554. DOI: 10.1371/journal.pone.0030554 From NLM Medline.
(84) Mao, X.; Kelty, T. J.; Kerr, N. R.; Childs, T. E.; Roberts, M. D.; Booth, F. W. Creatine supplementation upregulates mTORC1 signaling and markers of synaptic plasticity in the dentate gyrus while ameliorating LPS-induced cognitive impairment in female rats. Nutrients 2021, 13 (8), 2758.
(85) McMorris, T.; Mielcarz, G.; Harris, R. C.; Swain, J. P.; Howard, A. Creatine supplementation and cognitive performance in elderly individuals. Aging, Neuropsychology, and Cognition 2007, 14 (5), 517-528.
(86) Li, Z.; Wang, P.; Yu, Z.; Cong, Y.; Sun, H.; Zhang, J.; Zhang, J.; Sun, C.; Zhang, Y.; Ju, X. The effect of creatine and coenzyme q10 combination therapy on mild cognitive impairment in Parkinson's disease. European neurology 2015, 73 (3-4), 205-211.
(87) Hass, C. J.; Collins, M. A.; Juncos, J. L. Resistance training with creatine monohydrate improves upper-body strength in patients with Parkinson disease: a randomized trial. Neurorehabilitation and neural repair 2007, 21 (2), 107-115.
(88) Writing Group for the, N. E. T. i. P. D. I.; Kieburtz, K.; Tilley, B. C.; Elm, J. J.; Babcock, D.; Hauser, R.; Ross, G. W.; Augustine, A. H.; Augustine, E. U.; Aminoff, M. J.; et al. Effect of creatine monohydrate on clinical progression in patients with Parkinson disease: a randomized clinical trial. JAMA 2015, 313 (6), 584-593. DOI: 10.1001/jama.2015.120.
(89) Klivenyi, P.; Gardian, G.; Calingasan, N. Y.; Yang, L.; Beal, M. F. Additive neuroprotective effects of creatine and a cyclooxygenase 2 inhibitor against dopamine depletion in the 1-methyl-4-phenyl-1, 2, 3, 6-tetrahydropyridine (MPTP) mouse model of Parkinson's disease. Journal of Molecular Neuroscience 2003, 21, 191-198.
(90) Leem, Y. H.; Park, J. S.; Park, J. E.; Kim, D. Y.; Kim, H. S. Creatine supplementation with exercise reduces alpha-synuclein oligomerization and necroptosis in Parkinson's disease mouse model. J Nutr Biochem 2024, 126, 109586. DOI: 10.1016/j.jnutbio.2024.109586 From NLM Medline.
(91) Mazzini, L.; Balzarini, C.; Colombo, R.; Mora, G.; Pastore, I.; De Ambrogio, R.; Caligari, M. Effects of creatine supplementation on exercise performance and muscular strength in amyotrophic lateral sclerosis: preliminary results. Journal of the neurological sciences 2001, 191 (1-2), 139-144.
(92) Klivenyi, P.; Ferrante, R. J.; Matthews, R. T.; Bogdanov, M. B.; Klein, A. M.; Andreassen, O. A.; Mueller, G.; Wermer, M.; Kaddurah-Daouk, R.; Beal, M. F. Neuroprotective effects of creatine in a transgenic animal model of amyotrophic lateral sclerosis. Nature medicine 1999, 5 (3), 347-350.
(93) Andreassen, O. A.; Jenkins, B. G.; Dedeoglu, A.; Ferrante, K. L.; Bogdanov, M. B.; Kaddurah-Daouk, R.; Beal, M. F. Increases in cortical glutamate concentrations in transgenic amyotrophic lateral sclerosis mice are attenuated by creatine supplementation. Journal of neurochemistry 2001, 77 (2), 383-390.
(94) Dedeoglu, A.; Kubilus, J. K.; Yang, L.; Ferrante, K. L.; Hersch, S. M.; Beal, M. F.; Ferrante; J, R. Creatine therapy provides neuroprotection after onset of clinical symptoms in Huntington's disease transgenic mice. Journal of neurochemistry 2003, 85 (6), 1359-1367.
(95) Andreassen, O. A.; Dedeoglu, A.; Ferrante, R. J.; Jenkins, B. G.; Ferrante, K. L.; Thomas, M.; Friedlich, A.; Browne, S. E.; Schilling, G.; Borchelt, D. R. Creatine increases survival and delays motor symptoms in a transgenic animal model of Huntington's disease. Neurobiology of disease 2001, 8 (3), 479-491.
(96) Bender, A.; Auer, D. P.; Merl, T.; Reilmann, R.; Saemann, P.; Yassouridis, A.; Bender, J.; Weindl, A.; Dose, M.; Gasser, T.; et al. Creatine supplementation lowers brain glutamate levels in Huntington's disease. J Neurol 2005, 252 (1), 36-41. DOI: 10.1007/s00415-005-0595-4 From NLM Medline.
(97) Wilkinson, T. J.; Lemmey, A. B.; Jones, J. G.; Sheikh, F.; Ahmad, Y. A.; Chitale, S.; Maddison, P. J.; O'brien, T. D. Can creatine supplementation improve body composition and objective physical function in rheumatoid arthritis patients? A randomized controlled trial. Arthritis care & research 2016, 68 (6), 729-737.
(98) Roy, A.; Lee, D. Dietary creatine as a possible novel treatment for Crohn's ileitis. ACG Case Reports Journal 2016, 3 (4), e173.
(99) Chen, C. X.; Abdian, N.; Maussion, G.; Thomas, R. A.; Demirova, I.; Cai, E.; Tabatabaei, M.; Beitel, L. K.; Karamchandani, J.; Fon, E. A.; et al. A Multistep Workflow to Evaluate Newly Generated iPSCs and Their Ability to Generate Different Cell Types. Methods Protoc 2021, 4 (3). DOI: 10.3390/mps4030050 From NLM PubMed-not-MEDLINE.
(100) Krahn, A.; Reintsch, W.; Cai, E.; Durcan, T. M. Uptake of a-Synuclein Preformed Fibrils (PFFs) in Dopaminergic Neural Progenitor Cells. Zenodo 2020, (V1.0). DOI: https://doi.org/10.5281/zenodo.3875761.
(101) Chen, X. L., Nadine; Rocha, Cecilia; Rao, Trisha; Durcan, Thomas M. Generation of dopaminergic or cortical neurons from neuronal progenitors. Zenodo 2019. DOI: 10.5281/zenodo.3361000. X.-Q. Chen, C.; Deneault, E.; Abdian, N.; You, Z.; Sirois, J.; Nicouleau, M.; Shlaifer, I.; Villegas, L.; Boivin, M.-N.; Gaborieau, L.; et al. Generation of patient-derived pluripotent stem cell-lines and CRISPR modified isogenic controls with mutations in the Parkinson's associated GBA gene. Stem cell research 2022, 64, 102919. DOI: https://doi.org/10.1016/j.scr.2022.102919.
(102) Dorion, M. F.; Casas, D.; Shlaifer, I.; Yaqubi, M.; Fleming, P.; Karpilovsky, N.; Chen, C. X.; Nicouleau, M.; Piscopo, V. E. C.; MacDougall, E. J.; et al. An adapted protocol to derive microglia from stem cells and its application in the study of CSFIR-related disorders. Mol Neurodegener 2024, 19 (1), 31. DOI: 10.1186/s13024-024-00723-x From NLM Medline.
(103) DeGiosio, R. A.; Grubisha, M. J.; MacDonald, M. L.; Mckinney, B. C.; Camacho, C. J.; Sweet, R. A. More than a marker: potential pathogenic functions of MAP2. Front Mol Neurosci 2022, 15, 974890. DOI: 10.3389/fnmol.2022.974890 From NLM PubMed-not-MEDLINE.

1-20. (canceled)

21. A method of treating or preventing a neurodegenerative disorder selected from at least one of Alzheimer's disease, Parkinson's disease, Diffuse Lewy Body Disease, Multiple system atrophy, Amyotrophic Lateral Sclerosis, Frontotemporal Dementia, Corticobasal Degeneration, Progressive Supranuclear Palsy, Huntington's disease, or an autoimmune disorder selected from at least one of ankylosing spondylitis, Crohn disease, hidradenitis suppurativa, inclusion body myositis, juvenile idiopathic arthritis, plaque psoriasis, polyarticular juvenile idiopathic arthritis, psoriatic arthritis, rheumatoid arthritis, ulcerative colitis, and uveitis, the method comprising administering to a patient in need thereof a combination of (a) tauroursodeoxycholic acid (TUDCA) or ursodeoxycholic acid (UDCA), (b) coenzyme Q10 (CoQ10) as ubiquinone or ubiquinol, and (c) creatine.

22. The method of claim 21, wherein the dosage of TUDCA or UDCA is between 500 mg to 1200 mg per day, the dosage of CoQ10 is between 300 mg and 1200 mg per day, and the dosage of creatine is between 2500 mg and 7500 mg per day.

23. The method of claim 22, wherein the dosage of each of TUDCA or UDCA, CoQ10, and creatine is administered in one extended-release dosage form per day.

24. The method of claim 22, wherein the dosage of each of TUDCA or UDCA, CoQ10, and creatine is administered in one to four divided dosages per day.

25. The method of claim 22, wherein the combination is administered in one to six unit dosage forms per administration of the combination.

26. The method of claim 21, wherein the combination of (a) TUDCA or UDCA, (b) CoQ10, and (c) creatine is provided as one or more unit dosage forms selected from a tablet, capsule, and powder for dissolution in a beverage.
